(19) United States
(12) Patent Application Publication
(10) Pub. No.: US 2002/0137213 A1
(43) Pub. Date: Sep. 26, 2002

(54) Adenovirus particles with mutagenized fiber proteins

(76) Inventors: Paul L. Hallenbeck, Gaithersburg, MD (US); Neeraja Idamakanti, Gaithersburg, MD (US); John Leonard Jakubczak, Germantown, MD (US); Michael Kaleko, Rockville, MD (US); Michele Lynette Rollence, Damascus, MD (US); Theodore Smith, Ijamsville, MD (US); Susan C. Stevenson, Frederick, MD (US); David A. Stewart, Eldersburg, MD (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/870,203

(22) Filed: May 30, 2001

Related U.S. Application Data
(60) Provisional application No. 60/270,555, filed on Feb. 22, 2001.
(60) Provisional application No. 60/266,309, filed on Jun. 2, 2000.

(51) Int. Cl. C12N 15/861; C12N 5/06; C12N 7/00; C07K 14/075

(52) U.S. Cl. 435/456; 435/235.1; 530/350

(57) ABSTRACT

This invention relates to mutated adenoviral fiber proteins and adenovirus particles containing such proteins. It further relates to polynucleotides encoding the proteins and vectors containing polynucleotides. It also relates to methods for making and using the adenoviral particles. With the mutated fiber proteins, the adenovirus particles no longer bind to their natural cellular receptor. They can then be “retargeted” to a specific cell type through the addition of a ligand to the virus capsid, which causes the virus to bind to and infect such cell. Specific fiber mutations are listed, which ablate binding to the natural receptor. Adenovirus particles with certain fiber mutations were found to enhance gene transfer to and expression in liver as compared to viral particles with wild-type fiber.

This application claims the benefit under 35 USC § 119(e) of the following United States provisional applications: (1) Provisional Application No. to be assigned, filed Jun. 2, 2000 as application Ser. No. 09/585,344, and subject to a Petition for Conversion to Provisional Application filed Dec. 21, 2000; and (2) Provisional Application No. 60/270,555, filed Feb. 22, 2001. The disclosures of these applications are incorporated herein by reference in their entirety.

### BACKGROUND OF THE INVENTION

The wide tropism of adenoviral vectors is one of its advantages as a gene-delivery vehicle. However, there are a number of reasons why targeted vectors are desirable. Adenoviral vectors with increased transduction specificity should show reduced toxicity, since lower doses could be delivered to achieve the same desired therapeutic benefit. in addition, these lower doses should reduce potential immune responses to the viruses. This increased safety of targeted vectors would then allow for new routes of delivery, such as systemic administration, that would be applicable to a number of indications, like cancer and cardiovascular disease. Adenoviral particles with mutagenized fiber proteins are useful in the preparation of targeted adenoviruses.

### BRIEF DESCRIPTION OF THE FIGURES

FIG. 1 shows the strategy used for the production of pseudotyped adenoviral vectors with transiently expressed fiber proteins using the transient transfection/infection system.

FIG. 1A shows a schematic diagram of the genomic structure of Ad5.βgal.ΔF.

FIG. 1B shows the transient transfection/infection system. The fiber deleted adenoviral vector, Ad5.βgal.ΔF, as shown in panel A, can be grown in packaging cell lines transiently or stably expressing different fiber proteins to generate Ad5.βgal.ΔF/F⁺ fiber containing adenoviral particles. The vector is used to infect 293T cells that have been transfected with a fiber expression plasmid. The resulting particles will have new receptor tropisms dependent on the fiber protein.

FIG. 2 shows the differential fiber-dependent adenoviral transduction properties of HeLa cells using pseudotyped adenoviral vectors. HeLa cells were transduced with 1000 total particles per cell with the indicated pseudotyped adenoviral vector. After 24 hours, the cells were analyzed for β-galactosidase activity using a chemiluminescence reporter assay. The relative β-galactosidase activity of each pseudotyped adenoviral vector containing a mutated fiber protein was determined and normalized as a percentage of Ad5.βgal.ΔF/wt, which contains a wildtype fiber protein. All values are the mean percentage of Ad5.βgal.ΔF/wt, ±standard deviation (sd) derived from 5 to 6 separate transductions.

FIG. 3 is a plasmid map for p5FloxPRGD.

FIG. 4 is a plasmid map for pAv1h1pr.

FIG. 5 is a plasmid map for pSKO2, containing fiber mutations in combination with a cRGD targeting moiety.

FIG. 6 shows the transduction efficiency of adenovirus with retargeting ligand and detargeting fiber mutations. HDF (FIG. 6A), HeLa (FIG. 6B), CHO-K1 (FIG. 6C), and PC3 (FIG. 6D) cells were infected at 20 to 12500 total particles per cell in five-fold dose increments with the indicated fiber-modified adenoviral vectors. Av1nBg is the parental control with an unmodified fiber gene, Av1nBgHIRGD has been genetically altered to include cRGD in the HI loop, Av1nBgHIRGDKO1 has been genetically altered to include cRGD in the HI loop and the S408E, P409A mutation in fiber knob, and Av1nBgHIRGDKO2 has been genetically altered to include cRGD in the HI loop and the ΔV441, K442 mutation in fiber knob. After 24 hours, the cells were analyzed for β-galactosidase reporter gene activity using a chemiluminescence reporter assay.

FIG. 7 shows that Av1nBgHIRGDKO2 can compete transduction of HDF cells with Av1GFPHIRGD. HDF cells were infected at 1000 particles per cell with Av1GFPHIRGD, an adenoviral vector expressing GFP and which has been genetically altered to include cRGD in the HI loop. The infections were competed with Av1nBg, Av1nBgHIRGD, Av1nBgHIRGDKO1, and Av1nBgHIRGDKO2 at doses ranging from 1000 to 128,000 particles per cell in four-fold dose increments. After 24 hours, the cells were analyzed for GFP expression by measuring the fraction of cells that were positive for GFP expression by FACS analysis. The data was normalized as a percentage of Av1GFPHIRGD without competitor.

FIG. 8 is a plasmid map for pSKO1, containing fiber mutations in combination with a cRGD targeting moiety.

FIG. 9 is a plasmid map of pFLAv3nBgKO1 containing the full-length adenoviral genome with the KO1 fiber AB loop mutation.

FIG. 10 shows the transduction efficiency of Hela (FIG. 10A) and HDF (FIG. 10B) cells using adenoviral vectors containing fiber AB loop mutations.

FIG. 11 shows the transduction efficiency of Hep3B (FIG. 11A), HepG2 (FIG. 11B) and mouse hepatocytes (FIG. 11C) using adenoviral vectors containing fiber AB loop mutations.

FIG. 12 shows a competition viral transduction assay.

FIG. 13 shows in vivo adenoviral-mediated expression of P-galactosidase by an analysis of β-galactosidase activity in mouse livers.

FIG. 14 shows in vivo adenoviral-mediated transduction of mouse livers by hexon PCR analysis.

FIG. 15 shows in vivo adenoviral-mediated expression expression of β-galactosidase by an analysis of β-galactosidase activity in C57BL/6, Balb/C, and CD-1 mouse livers.

FIG. 16 shows in vitro adenoviral-mediated transduction of isolated primary CD-1 mouse hepatocytes.

### DESCRIPTION OF THE INVENTION

This invention relates to mutated adenoviral fiber proteins and adenovirus particles containing such proteins. It further relates to polynucleotides encoding the proteins and vectors containing the polynucleotides. It also relates to methods for making and using the adenoviral particles. With the mutated fiber proteins, the adenovirus particles no longer bind to their natural cellular receptor. They can then be “retargeted” to a specific cell type through the addition of a ligand to the virus capsid, which causes the virus to bind to and infect such cell.

As used herein, the term “adenovirus” or “adenoviral particle” is used to include any and all viruses that may be categorized as an adenovirus, including any adenovirus that infects a human or an animal, including all groups, subgroups, and serotypes. Preferably, such adenoviruses are ones that infect human cells. Such adenoviruses may be wild-type or may be modified in various ways known in the art or as disclosed herein. Such modifications include modifications to the adenovirus genome that is packaged in the particle in order to make an infectious virus. Such modifications include deletions known in the art, such as deletions in one or more of the E1, E2a, E2b, E3, or E4 coding regions. Such modifications also include deletions of all of the coding regions of the adenoviral genome. Such adenoviruses are known as “gutless” adenoviruses. The terms also include replication-conditional adenoviruses; that is, viruses that replicate in certain types of cells or tissues but not in other types. These include the viruses disclosed in U.S. Pat. No. 5,998,205, issued Dec. 7, 1999 to Hallenbeck et al. and U.S. Pat. No. 5,801,029, issued Sep. 1, 1998 to McCormick, the disclosures of both of which are incorporated herein by reference in their entirety. Such viruses are sometimes referred to as cytolytic or cytopathic viruses (or vectors), and, if they have such an effect on neoplastic cells, are referred to as oncolytic viruses (or vectors).

In one embodiment, the mutated adenoviral fiber protein of the invention is a fiber protein where at least one amino acid in the CD loop of a wild-type fiber protein of an adenovirus from subgroup C, subgroup D, subgroup E, or selected viruses from subgroup F, (in particular those having the long fiber) have been mutated to reduce or substantially eliminate the ability of the fiber protein to bind to the cellular receptor known as the coxsackievirus-adenovirus receptor (CAR) to which the wild-type fiber of these subgroups, as well as subgroup A, bind. These subgroups are standard taxonomic designations known to those skilled in the art. Subgroup A includes adenovirus serotypes 12, 18, and 31. Subgroup C includes adenovirus serotypes 1, 2, 5, and 6. Subgroup D includes adenovirus serotype 8, 9, 10, 13, 15, 17, 19, 20, 22-30, 32, 33, 36-39, and 42-49. Subgroup E includes adenovirus serotype 4. Subgroup F includes adenovirus serotypes 40 and 41. These latter two serotypes have both a long and a short fiber protein. Only the long fiber protein binds to CAR. The preferred adenovirus serotype of the invention is adenovirus serotype 5.

The reduction or elimination of the ability of the mutated adenovirus fiber protein to bind CAR as compared to the corresponding wild-type fiber protein is measured by comparing the transduction efficiency (gene transfer and expression of a marker gene) of an adenovirus particle containing the mutated fiber protein compared to an adenovirus particle containing the wild-type fiber protein for cells having CAR. As used herein, the term “substantially eliminate” refers to a transduction efficiency less than about 11% of the efficiency of the wild-type fiber containing virus on Hela cells using the transient transfection/infection system described in Example 1. Preferably, the efficiency is less than a b o u t 9%. Most preferably, the efficiency is less than about 8%. As used herein, the phrase “reduce” or “reduction” refers to a change in the efficiency of transduction by the adenoviruses containing the mutated fiber as compared to the adenovirus containing the wild-type fiber to a levl of about 75% or less of the wild-type on Hela cells using the transient transfection/infection system described in Example 1. Preferably, the change in efficiency is to a level of about 65% or less than wild-type. Most preferably, it is about 55% or less.

The fiber proteins of the invention are modified by chemical and biological techniques known to those skilled in the art. Such techniques permit the mutation of at least one amino acid in the CD loop of the wild-type fiber protein to change the ability of the protein to bind to CAR. As used herein, the term “mutate” or “mutation” or similar terms refers to the deletion or change of at least one amino acid in this part of the protein. The amino acid can be changed by substitution or by modification in a way that derivatizes the amino acid.

As mentioned above, the preferred fiber protein of the invention is a mutated adenovirus serotype 5 fiber protein. The amino acid sequence of the wild-type protein is shown in SEQ ID NO:2. The CD loop in the wild-type adenovirus 5 protein extends from the amino acid at position 441 to the amino acid at position 453. Preferably, the amino acid at position 441 and/or the amino acid at position 442 of the wild-type fiber protein is mutated. Such mutation may involve a deletion of the amino acid at either or both of positions 441 and 442 (SEQ ID NOS:6, 10, 12, 13). Alternatively, substitution at either or both of these positions may be made. In a particularly preferred embodiment, the amino acid at position 441 of the wild-type fiber protein is changed from valine to alanine. In another particularly preferred embodiment, the amino acid at position 442 of the wild-type fiber protein is changed from lysine to alanine. Most preferably, the amino acid at position 441 of the wild-type fiber protein is changed from valine to alanine, and the amino acid at position 442 of the wild-type fiber protein is changed from lysine to alanine (SEQ ID NO: 14).

The present inventors have also discovered that certain mutations in other parts of the wild-type adenovirus 5 fiber protein reduce or substantially eliminate the ability of an adenoviral particle with the mutated fiber to bind to CAR. In a preferred embodiment, the mutations are at one or more of amino acid positions 408, 409, 460, 509, 510, 538, and 539 of the wild-type protein. In one particularly preferred embodiment, the fiber protein is mutated at amino acid positions 408 and 409, preferably by substituting glutamic acid for serine at position 408 and substituting alanine for proline at position 409 (SEQ ID NO:4). In another preferred embodiment, the fiber protein is mutated at amino acid position 460 of the wild-type fiber protein, most preferably by substituting glutamic acid for arginine (SEQ ID NO:16). In another preferred embodiment, the fiber protein is mutated at at least one of amino acid positions 509 and 510 of the wild-type fiber protein, preferably by deleting the Case No. 4-31452A/GTI amino acids at both positions (SEQ ID NO:18). In another preferred embodiment, the fiber protein is mutated at at least one of amino acid positions 538 and 539 of the wild-type fiber protein, preferably by deleting the amino acids at both positions (SEQ ID NO:20).

Any or all of these mutations may be combined with mutations in the CD loop of adenovirus 5. In a preferred embodiment, the mutated fiber protein of the invention comprises at least one mutation at amino acid positions 441 and 442 of the wild-type fiber protein plus a mutation at one or more of amino acid positions 408, 409, 460, 509, 510, 538, and 539 of the wild-type fiber protein. For example, SEQ ID NO:8.

In an alternative, preferred embodiment, the mutated adenoviral fiber protein of the invention is a fiber protein where at least one amino acid in the AB loop of a wild-type fiber protein of an adenovirus from subgroup C, subgroup D, subgroup E, or selected viruses from subgroup F (in particular those having a long fiber), have been mutated to reduce or substantially eliminate the ability of the fiber protein to bind to CAR. In this embodiment, the preferred fiber protein of the invention is a mutated adenovirus serotype 5 fiber protein.

More preferably, the mutated adenovirus serotype 5 fiber protein contains mutations at amino acid positions 408 and/or 409 of the wild-type fiber protein. Preferably, the mutations are at both positions. As mentioned previously, such mutations may be deletions, substitutions, or a modification in a way that derivitizes the amino acid. The same type of mutation need not be made at each position. In one preferred version of this preferred embodiment, glutamic acid is substituted for serine at position 408. In an alternative preferred version of this preferred embodiment, alanine is substituted for proline at position 409. Most preferably, glutamic acid is substituted for serine at position 408, and alanine is substituted for proline at position 409 (SEQ ID NO:4).

The invention also comprises polynucleotides that encode the proteins of the invention. As used herein, the term “polynucleotide” means a nucleic acid molecule, such as DNA or RNA, that encodes a polynucleotide. The molecule may include regulatory sequences. Preferably, the polynucleotide is DNA. Such polynucleotides are prepared or obtained by techniques known by those skilled in the art in combination with the teachings contained therein. Examples of such polynucleotides are shown in SEQ ID NOS:1, 3, 5, 7, 9, 11, 13, 15, 17, 19. The polynucleotides of the invention also include polynucleotides that differ in certain bases but still encode the proteins of the invention due to the redundancy of the genetic code.

The invention further comprises vectors including the polynucleotides of the invention. Such vectors include partial or complete adenoviral genomes and plasmids. Such vectors are constructed by techniques known to those skilled in the art.

One of the principal uses of such vectors is the production of adenoviral packaging cells. The packaging cells of the invention are cells that provide complementing functions to the functions provided by the genes in the adenovirus genome that are to be packaged into the adenovirus particle. The production of such particles require that the genome be replicated and that those proteins necessary for assembling an infectious virus be produced. The particles may also require certain proteins necessary for the maturation of the viral particle. Such proteins may be provided by the vector or by the packaging cell.

The packaging cells of the invention may contain the polynucleotide encoding the mutated fiber protein. Such polynucleotide may be transfected into the cell, preferably as part of a plasmid, or it may be infected into the cell with a viral vector. It may be stably incorporated into the genome of the cell, thus providing for a stable cell line. Alternatively, it may be unincorporated into the genome, in which case a transient complementing cell will be provided.

The adenovirus genome to be packaged is transferred into the complementing cell by techniques known to those skilled in the art. These techniques include transfection or infection with another virus. The polynucleotide encoding the mutated fiber protein may be in this genome instead of in the packaging cell.

In certain cases, it may be desirable, when the polynucleotide encoding the mutated fiber is in the genome to be packaged, for the packaging cell to also encode a fiber protein. Such protein may assist in the maturation and packaging of an infectious particle. Such protein may be a wild-type fiber protein or one modified so as to be unable to attach to the penton base protein.

The packaging cells are cultured under conditions that permit the production of the desired viral particle. The viral particles are recovered by standard techniques.

A preferred way of making the adenoviral particles of the invention is as follows. The polynucleotide encoding the mutated fiber protein is made using standard techniques in an adenoviral shuttle plasmid. This plasmid contains the right end of the virus, in particular from the end of the E3 region through the right ITR. It also contains a recombinase site, such as a lox site. This plasmid is co-transfected into a complementing cell line along with a helper plasmid, which contains the remaining portion of the adenovirus genome, except for the E1 region and sometimes also the E2a region. A third plasmid, which is an expression plasmid containing a gene encoding a recombinase such as Cre, is also transfected into the complementing cell. The complementing cell is preferably a 293 cell, which contains the adenoviral E1 genes, or an AE1-2a cell {Gorziglia, Kadan, et al. 1996}, which contains the adenoviral E1 and E2a genes. Most preferably, the complementing cell is a 633 cell {Von Seggern, Huang, et al. 2000}, which stably expresses the adenovirus serotype 5 wild-type fiber protein, and was derived from the AE1-2a cell line.

The transfected complementing cells are maintained under standard cell culture conditions. The adenoviral plasmids recombine to form the adenoviral genome that is packaged. The particles are infectious, but replication deficient because their genome is missing at least the E1 genes. The particles contain both wild-type and mutated fiber proteins. They are recovered from the crude viral lysate and are purified by standard techniques.

The recovered particles are preferably used to infect 293 or AE1-2a cells. This permits the recovery of particles whose capsids contain only the desired mutated fiber. This two-step procedure provides high titer batches of the adenoviral particles of the invention.

The adenoviral particles may be replication competent or replication incompetent. In a preferred embodiment of the invention, the particles selectively replicate in certain predetermined target tissue but are replication incompetent in other cells and tissues. In a particularly preferred embodiment of the invention, the adenoviral particles replicate in abnormally proliferating tissue, such as solid tumors and other neoplasms. Such replication conditional adenoviral particles and vectors may be produced by techniques known to those skilled in the art, such as those disclosed in the above-referenced U.S. Pat. Nos. 5,998,205 and 5,801,029. These particles and vectors may be produced in adenoviral packaging cells as disclosed above. The preferred packaging cells are those that have been designed to limit homologous recombination that could lead to wild-type adenoviral particles. Such cells are disclosed in U.S. Pat. No. 5,994,128, issued Nov. 30, 1999 to Fallaux, et al., and U.S. Pat. No. 6,033,908, issued Mar. 7, 2000 to Bout, et al. The packaging cell known as PER.C6, which is disclosed in these patents, is particularly preferred.

Preferably, the modified fiber polynucleotide also includes sequences that encode a targeting ligand. Accordingly, such sequences are transfected into the complementing cell by the shuttle plasmid.

Alternatively, the targeting ligand sequences may be included in the penton or hexon proteins. In such cases, they would be in the helper plasmid.

The packaged adenoviral genome may also contain a heterologous polynucleotide. This polynucleotide is usually included in the deleted E1 region of the helper plasmid. Alternatively, the polynucleotide may be in the E3 region, in which case it is included in the shuttle plasmid.

The adenovirus particles of the invention include the mutated fiber proteins. Such particles may include different types of the mutated fibers of the invention. They may also include wild-type fibers along with the mutated fibers.

The adenoviral particles of the invention preferably further comprise a targeting ligand included in a capsid protein of the particle. The ligand may be included in any of the capsid proteins, such as fiber, hexon, or penton. Preferably, the ligand is included in a fiber protein, which is preferably a mutated fiber protein of the invention. Most preferably, the targeting ligand is included within the HI loop of the fiber protein. Any ligand that can fit in the HI loop and still provide a functional virus may be used. Such ligands may be as long as 80-90 amino acids. Such ligands are added by techniques known in the art, such as those disclosed in PCT application PCT/US99/02549, published as WO99/39734 on Aug. 12, 1999, and Example 12 of U.S. patent application Ser. No. 09/482,682, filed Jan. 14, 2000, the disclosures of both of which are hereby incorporated herein by reference.

A targeting ligand may be any chemical moiety that preferentially directs the adenoviral particle to a desired cell type. The categories of such ligands include peptides, polypeptides, single chain antibodies, and multimeric proteins. Specific ligands include the TNF superfamily of ligands which include tumor necrosis factors (or TNF's) such as, for example, TNF-alpha and TNF-beta, lymphotoxins (LT), such as LT-αand LT-β, Fas ligand which binds to Fas antigen; CD40 ligand, which binds to the CD40 receptor of B-lymphocytes; CD30 ligand, which binds to the CD30 receptor of neoplastic cells of Hodgkin's lymphoma; CD27 ligand, NGF ligand, and OX-40 ligand; transferrin, which binds to the transferrin receptor located on tumor cells, activated T-cells, and neural tissue cells; ApoB, which binds to the LDL receptor of liver cells; alpha-2-macroglobulin, which binds to the LRP receptor of liver cells; alpha-1 acid glycoprotein, which binds to the asialoglycoprotein receptor of liver; mannose-containing peptides, which bind to the mannose receptor of macrophages; sialyl-Lewis-X antigen-containing peptides, which bind to the ELAM-1 receptor of activated endothelial cells; CD34 ligand, which binds to the CD34 ligand, which binds to the CD34 receptor of hematopoietic progenitor cells; ICAM-1, which binds to the LFA-1 (CD11b/CD18) receptor of lymphocytes, or to the Mac-1 (CD11a/CD18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-1 receptor of activated endothelial cells; HIV gp120 and Class II MHC antigen, which bind to the CD4 receptor of T-helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; Interleukins 1 through 14, which bind to the Interleukin 1 through 14 receptors, respectively; the Fv antigen-binding domain of an immunoglobulin; and cyclic RGD peptide. Cyclic RGD (cRGD) is preferred. As used herein, the term “cyclic RGD” (or cRGD) refers to any amino acid that binds to a integrins on the surface of cells and contains the sequence RGD (Arg-Gly-Asp). The sequence in SEQ ID NO:43 is particularly preferred.

In a preferred embodiment, because the adenovirus fiber has a trimeric structure, the ligand also has a trimeric structure. In a more preferred embodiment, the ligand is selected from the TNF superfamily of ligands hereinabove described. Such ligands are trimeric and of similar size to the fiber head domain. Such ligands may be incorporated into the fiber protein using the techniques disclosed in U.S. Pat. No. 5,756,086, issued May 26, 1998 to McClelland et al., the disclosure of which is incorporated herein by reference.

The adenovirus particles may further include at least one heterologous polynucleotide. As used herein, the term “heterologous polynucleotide” means a polynucleotide derived from a biological source other than an adenovirus, which encodes a polypeptide when the adenovirus infects a cell. Such polynucleotides are included in the adenoviral genome within the particle and are added to that genome by techniques known in the art. Any heterologous polynucleotide of interest may be added. A preferred polynucleotide is one that encodes an immunostimulating protein, such as an interleukin, interferon, or colony stimulating factor. Mammalian GM-CSF is preferred. Preferably, such GM-CSF is a primate GM-CSF; most preferably, it is human GM-CSF. An alternative preferred polynucleotide encodes herpes simplex virus thymidine kinase (HSV-TK), which is useful as a safety switch as described in U.S. patent application Ser. No. 08/974,391, filed Nov. 19, 1997, which published as PCT Publication No. WO/9925860, the disclosure of which is incorporated herein by reference.

The adenoviral particles of the invention are used to genetically engineer a cell to express a protein that it otherwise does not express or does not express in sufficient quantities. This is accomplished by infecting the desired cell with an adenoviral particle of the invention whose genome includes a desired heterologous polynucleotide. This permits the expression of the heterologous polynucleotide in the cell. Preferably, the cell is a mammalian cell. More preferably, the mammalian cell is a primate cell. Most preferably, the primate cell is a human cell. The cell may be inside the body of the animal (in vivo) or outside the body (in vitro).

Preferably, the adenoviral particle includes a targeting ligand as described above. This permits the delivery of a gene to a desired cell type which is different from the cell type that wild-type adenovirus particle infect or the same as a wild-type particle would infect but allowing the infection in a selective manner, i.e., non-target cell types would not be infected.

Thus, the adenoviruses of the invention can be used to study cell transduction and gene expression in vitro or in various animal models. The latter case includes ex vivo techniques, in which cells are transduced in vitro and then administered to the animal. They may also be used to conduct gene therapy on humans or other animals. Such gene therapy may be ex vivo or in vivo. For in vivo gene therapy, the adenoviral particles of the invention in a pharmaceutically-acceptable carrier are delivered to a human in a therapeutically effective amount in order to prevent, treat, or ameliorate a disease or other medical condition in the human through the introduction of a heterologous gene that encodes a therapeutic protein into cells in such human. The adenoviruses are delivered at a dose ranging from approximately 1 particle per kilogram of body weight to approximately 10¹⁴ particles per kilogram of body weight. Preferably, they are delivered at a dose of approximately 10⁶ particles per kilogram of body weight to approximately 10¹³ particles per kilogram of body weight. Most preferably, the dose ranges from approximately 10⁹ particles per kilogram of body weight to approximately 10¹² particles per kilogram of body weight.

The adenoviral particles of the invention with the above-identified modifications in the AB loop, particularly those with modifications at amino acid positions 408 and 409 of the wild-type adenovirus serotype 5 fiber protein, and most particularly those where glutamic acid is substituted for serine at position 408 and alanine is substituted for proline at position 409 (SEQ ID NO:4), have additional desirable utilities. The inventors have unexpectedly discovered that such viral particles provide enhanced gene transfer to and expression in hepatocytes in the liver of an animal as compared to adenoviral particles with the wild-type fiber protein. Therefore, the invention includes a method of enhancing adenoviral-mediated gene transfer to and expression in cells in the liver of an animal by administering adenoviruses having such AB loop modification in at least one of their fiber proteins to an animal under conditions where cells in the liver are transduced. The hepatocytes are the cells that are primarily transduced. Preferably, an adenovirus particle comprising a mutated adenovirus serotype 5 fiber protein, wherein glutamic acid is substituted for serine at amino acid position 408 and alanine is substituted for proline at amino acid position 409 (SEQ ID NO:4), is used to deliver the heterologous gene.

Such adenoviral particles would be particularly useful for gene therapy where it is desired to express a heterologous gene in a patient's liver. This could be used, for example, in the treatment of diabetes, hemophilia, and diseases related to increased cholesterol or triglyceride blood levels in a patient such as atherosclerosis. It would also include anti-angiogenesis treatment methods involving the delivery of one or more anti-angiogenic genes to the hepatocytes of a patient's liver.

Because of the enhanced gene transfer and expression, lower doses of these viral particles would be able to be used. The dose for these types of particles would be approximately 1 particle per kilogram of body weight to approximately 10¹³ particles per kilogram of body weight. Preferably, the dose would be approximately 10⁵ particles per kilogram of body weight to approximately 10¹² particles per kilogram of body weight. Most preferably, the dose ranges from approximately 10⁸ particles per kilogram of body weight to approximately 10¹¹ particles per kilogram of body weight.

Such particles are delivered by routes of administration known to those skilled in the art. One such route is intravenous injection. An alternative route is intraparenchymal injection. The particles may also be delivered by injection into the hepatic artery, portal vein, or bile duct.

Another use of the particles with the AB loop mutations would be as a basis for further modification of the particles, wherein the RGD amino acid sequence in the penton protein is deleted, modified, or substituted. Techniques for such modifications are disclosed in U.S. Pat. No. 5,559,099, issued Sep. 24, 1996 to Wickham, et al., U.S. Pat. No. 5,712,136, issued Jan. 27, 1998 to Wickham, et al., and U.S. Pat. No. 5,731,190, issued Mar. 24, 1998 to Wickham, et al., the disclosures of which are incorporated herein by reference. This would prevent the particles from binding to cell surface integrins and being taken into the cell.

### EXAMPLES

### Example 1

### Adenovirus Type 5 Viral Particles Pseudotyped With Mutagenized Fiber Proteins Show Diminished Infectivity of Coxsackie B-Adenovirus Receptor-Bearing Cells

Introduction

The great interest in human adenovirus type 5 (AdS) as a gene delivery platform is due in part to its ability to efficiently infect many cell types. Its wide tropism is mediated by a primary interaction between the Ad5 capsid protein, fiber, and its high-affinity cellular receptor, the coxsackie adenovirus receptor (CAR). Fiber is a homotrimeric protein present twelve times on the viral capsid. It has three domains: an N-terminal tail that interacts with the penton base in the viral capsid, a rod-like shaft containing 22 copies of a 15 amino-acid beta sheet structure, and a globular knob domain. It is the knob domain that mediates binding to CAR during cell attachment. After the initial binding event, a second, low-affinity interaction takes place between the penton base and αᵥ integrins on the cell surface. This step is required for virus internalization and subsequent gene transfer.

There are many cases where it is desirable to deliver therapeutic genes to a subset of cell types. For this reason, there has been much effort to specifically target Ad5 vectors. This capability involves the detargeting away from its natural receptor and the simultaneous retargeting of the viral tropism toward a given cell type. The resulting vector would represent an important step in the development of this gene therapy platform, both from an efficacy and a safety standpoint. Reducing the undesired virus-tissue interactions and increasing the intended interaction would allow lower viral doses to be used and thereby potentially minimize the associated toxic side effects and host immune response.

Several strategies have been used to alter the receptor tropism and binding specificity of adenoviral vectors. These strategies include replacing the fiber knob domain with a knob from another Ad serotype with a different receptor specificity {Stevenson, Rollence, et al. 1995} {Krasnykh, Mikheeva, et al. 1996} {Stevenson, Rollence, et al. 1997}, the insertion of peptides onto the C-terminus of fiber {Wickham, Granados, et al. 1990} {Michael, Hong, et al. 1995} {Wickham, Tzeng, et al. 1997} or the exposed HI loop {Krasnykh, Dmitriev, et al. 1998} and the use of bifunctional antibodies {Wickham, Segal, et al. 1996}. The results of these efforts have been an expansion of viral tropism, which is suitable for some gene therapy applications such as vascular gene therapy where the aim is to improve the gene transfer efficiency of Ad vectors that are delivered locally. However, to specifically transduce certain cell types with systemically-delivered adenoviral vectors, it will be necessary to ablate the natural receptor tropism in combination with the introduction of a high affinity targeting ligand.

The analysis of multiple fiber mutations in the context of a viral particle remains a tedious process that involves the time-consuming incorporation of modified fiber genes into the adenoviral genomic DNA. Furthermore, the incorporation of mutated fiber genes into the Ad genome may affect the efficient growth and propagation of Ad. As a result, the generation and evaluation of adenoviral vectors containing mutated fiber proteins may require alternative means of growing the vectors that will allow for the efficient production of high titer viral stocks.

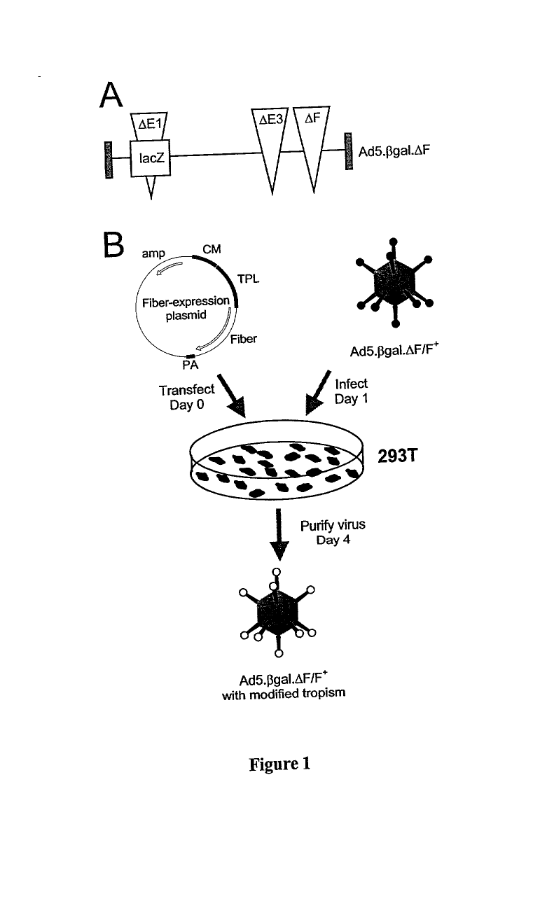

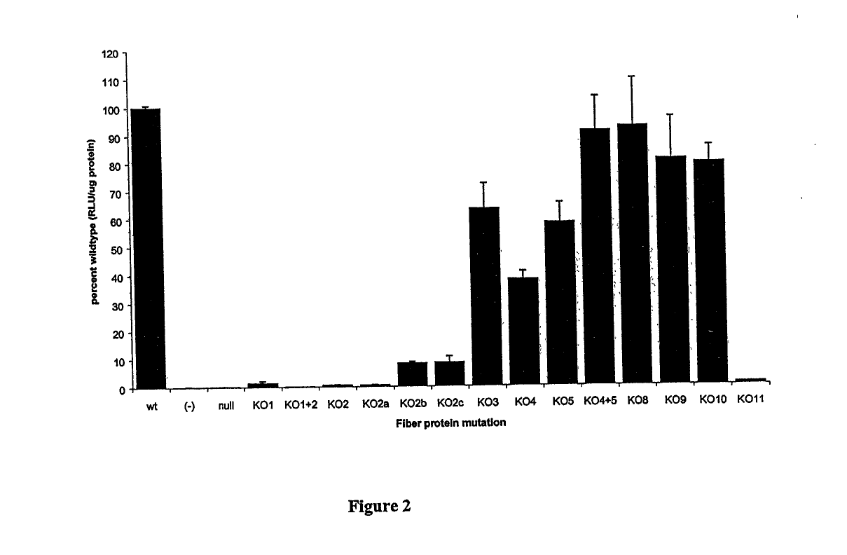

We have developed a novel system to rapidly analyze modified fiber proteins for desired tropism in the context of the viral particle. This system is based on the ability to pseudotype a fiberless Ad5 mutant with fiber proteins expressed transiently from an episomal plasmid (FIG. 1). The fiber-deleted Ad vector is Ad5.gal.ΔF, which is an E1-E3- and fiber-gene deleted adenovirus that expresses cytoplasmic β-galactosidase under the control of the SV40 promoter {Von Seggern, Chiu, et al. 1999} (FIG. 1A). The modified fiber proteins for pseudotyping are produced from expression plasmid constructs designed for high levels of fiber protein expression {Von Seggern, Huang, et al. 2000}. The primary advantage of this system is that modified fiber proteins can be quickly incorporated into virions and functionally analyzed in their most relevant context for their effect on CAR interaction and subsequent gene transfer and expression. We used this system to analyze a panel of fiber mutants for their ability to mediate adenoviral gene transfer to Hela cells, a CAR-expressing cell line. We have shown that the transient transfection/infection system can efficiently pseudotype a fiberless viral capsid with levels of fiber protein indistinguishable from those seen on wildtype virions. We used this system to identify multiple fiber gene mutations that significantly reduce the ability of adenovirus to transduce cells and achieve gene transfer.

Materials and Methods

Plasmids and Fiber Gene Mutagenesis.

The Ad5 fiber cDNA has been cloned into pcDNA3.1 to generate pDV60, as previously described {Von Seggern, Huang, et al. 2000}. Briefly, pDV60 contains the CMV promoter, the first Ad5 tripartite leader exon (TPL), the natural first intron and the fused second and third TPL exons upstream of the Ad5 fiber gene. All amino acid changes were incorporated into the fiber cDNA using the pDV60 plasmid as the template. Individual amino acid residues in pDV60 were mutagenized using the QuickChange Site-Directed Mutagenesis system (Stratagene, La Jolla Calif.). The oligonucleotide primers used for the incorporation of amino acid changes are listed in Table 1 for each single or double amino acid modification. The thermal cycler protocol was 95° C. for 30 sec, followed by 18 cycles of 95° C. for 30 sec, 55° C. for 1 min, and 68° C. for 20 min.

The entire knob domain of the Ad5 fiber was deleted from amino acids 404 to 581. For restoration of trimerization of the fiber tail and shaft, a 31 amino acid peptide derived from the GCN4 leucine zipper {Harbury, Zhang, et al. 1993} was fused immediately after the fiber TLWT sequence at the fiber shaft-head junction using PCR gene overlap extension {Horton, Cai, et al. 1990}. This reaction fused the Ad5 fiber tail and shaft regions (amino acids 1 to 403) to the GCN4 isoleucine 31 amino acid peptide to form the KO11 mutant and was cloned into pDV60 to create pDKO11. For all fiber mutations, the nucleotide sequence of the cloned insert was determined and in each case a clone having the expected sequence was selected. The pDV55 control plasmid is similar to pDV60, except that it lacks the fiber gene {Von Seggem, Huang, et al. 2000}.

Viruses.

Ad5.βgal.wt is a first generation E1-, E3-deleted adenovirus containing a lacZ reporter cassette in the E1 region {Von Seggern, Chiu, et al. 1999}. Ad5.βgal.ΔF is identical to Ad5.βgal.wt except that the fiber gene is deleted {Von Seggern, Chiu, et al. 1999}.

Cells.

Human 293T cells were obtained from ATCC (CRL 11268) and were cultured in the DMEM containing 10% FBS. The 293T cells stably express the SV40 large T antigen that allows for the amplification of plasmids from the SV40 origin of replication. The 633 cells stably express the Ad5 fiber protein {Von Seggern, Huang, et al. 2000} and are derived from AE1-2a, a cell line that complements E1a- and E2a-deleted adenoviral vectors {Gorziglia, Kadan, et al. 1996}. 633 cells were grown in Richter's CM (Life Technologies #C-2671) and 10% FBS. Hela cells (ATCC CCL-2) were grown in Dulbecco's modified Eagle's media supplemented with 10% FBS. For a further description of the preparation of AE1-2a cells, also known as S8 cells, and the preparation of 633 cells, see Example 6F and 6G of U.S. patent application Ser. No. 09/482,682, filed Jan. 14, 2000, which disclosure is incorporated herein by reference.

Transient Transfection/Infection.

Mutated fiber proteins were incorporated into adenoviral particles using the transient transfection/infection system. For each virus preparation using the transient transfection/infection system, four 15 cm dishes of 70% confluent 293T cells were used. For transfections, 100 ug of each fiber expression plasmid DNA listed in Table 2, 400 ul lipofectamine (Life Technologies, Rockville, Md.) and 3.6 ml Opti-MEM 1 media (Life Technologies, Rockville, Md.) were combined in a conical 250 ml sterile bottle. At the end of a 30 minute room temperature incubation, 60 ml Opti-MEM 1 media was added. A 16 ml aliquot of this transfection mix was added to each plate and incubated at 37° C., 5% CO₂ for 5 hours. The transfection media was then aspirated and 20 ml of complete DMEM media was added. The dishes were then incubated at 37° C., 5% CO₂ for 24 hours to allow expression of the fiber protein.

The transfected 293T cells were then infected with Ad5.βGal.AF/F⁺ virus at a particle per cell ratio of 350. The Ad5.βGal.AF/F⁺ virus is an E1, E3, fiber-deleted AdS vector {Von Seggem, Chiu, et al. 1999} that was propagated in the fiber-complementing cell line, 633, such that the capsid contains wildtype Ad5 fiber protein {Von Seggern, Huang, et al. 2000}. The growth media was aspirated and 2.5 ml of infection media (DMEM and 2% FBS) containing Ad5.βGal.AF/F⁺ was added and slowly rocked at 37° C., 5% CO₂ for 2 hours. Twenty ml of growth media was then added (DMEM and 10% FBS) and the plates were incubated at 37° C., 5% CO₂ overnight. The media was replaced the next day and the incubation was continued until complete cytopathic effect (CPE) was observed, typically in about 3 to 4 days. The transfected/infected 293T cells were harvested after complete CPE by gently dislodging the cells, pelleting by centrifugation, and resuspending in lml phosphate buffered saline. A crude viral lysate (CVL) was prepared by five freeze-thaw cycles to disrupt the cells and release the virus. The virus was purified by CsCl gradient centrifugation using standard procedures. The virus particle titer was determined spectrophotometrically as described {Mittereder, March, et al. 1996}. Yields of Ad5.βGal.ΔF virus pseudotyped with modified fiber protein typically ranged from 10¹¹ to 10¹² particles.

Western Immunoblot Analysis.

The expression and incorporation of each fiber protein onto adenoviral particles was verified by denaturing sodium dodecyl-sulfate (SDS) 4 to 12% polyacrylamide gel electrophoresis (PAGE) and Western immunoblot analysis. An aliquot of each adenoviral vector preparation corresponding to 5.0×10⁹ particles per lane was analyzed. The proteins were transferred to a nitrocellulose membrane with a minitransblot apparatus (Novex Inc.) for 90 minutes at 30V. The membrane was blocked for at least 1 hour at room temperature in lOmM Tris, pH7.4 containing 150 mM NaCl, 2 mM EDTA, 0.04% Tween-20, and 5% dried milk. The blocked membrane was incubated for 1 hour with a 1:1000 dilution of a primary rabbit anti-Ad5 fiber polyclonal antiserum. The membrane was then developed with a 1:5000 dilution of the secondary donkey anti-rabbit IgG horseradish peroxidase-conjugated antibody (Amersham Lifesciences, Arlington Heights, Ill.) using an enhanced chemiluminescense system (Amersham Lifesciences). The membrane was exposed to film for approximately 1 to 20 seconds. The membrane was then used to reprobe for detection of the adenoviral penton protein to ensure equivalent loading of viral particles. Briefly, the membrane was incubated for 1 hour with a 1:5000 dilution of the primary rabbit anti-Ad5 penton polyclonal antiserum. The membrane was then re-developed with a 1:5000 dilution of the secondary goat anti-rabbit IgG horseradish peroxidase-conjugated antibody as described above.

Production of Anti-Ad5 Fiber and Anti-Ad5 Penton-Specific Antiserum.

Both of the rabbit primary antibodies used in the anti-fiber and anti-penton Western immunoblot analysis were generated by immunizations of New Zealand White rabbits (Loftstrand Labs, Ltd., Gaithersburg, Md.). The Ad5 fiber and penton proteins were expressed using the baculoviral expression system. The purified Ad5 fiber protein and partially purified penton base proteins were used for immunizations according to standard protocols. The antiserum obtained was tested for immunoreactivity against the Ad5 fiber and penton proteins by Western immunoblot analysis.

Adenoviral Transduction.

Hela cells were infected with the adenoviral vectors containing mutated fiber proteins to evaluate the effects of fiber amino acid mutations on CAR interaction and subsequent gene expression. Monolayers of HeLa cells in 12 well dishes were infected with 1000 particles per cell for 2 hours at 37° C. in a total volume of 0.35 ml of the DMEM containing 2% FBS. The infection medium was then aspirated from the monolayers and lml of complete DMEM containing 10% FBS was added per well. The cells were incubated for an additional 24 hours to allow for β-galactosidase expression.

β-Galactosidase Expression Analysis.

The expression of β-galactosidase encoded by the adenoviral vectors in the infected cells was measured by a chemiluminescence reporter assay and by histochemical staining with a chromogenic substrate. The relative levels of β-galactosidase activity were determined using the Galacto-Light chemiluminescence reporter assay system (Tropix, Bedford, Mass.). Briefly, the cell monolayers were washed with PBS and processed according to the manufacturer's protocol. The cell homogenate was transferred to a microfuge tube and centrifuged to remove cellular debris. Total protein concentration was determined using the bicinchoninic acid (BCA) protein assay (Pierce, Inc., Rockford, Ill.) with bovine serum albumin as the assay standard. An aliquot of each sample was then incubated with the Tropix β-galactosidase substrate for 45 minutes in a 96 well plate. A luminometer was used to determine the relative light units (RLU) emitted per sample and then normalized for the amount of total protein in each sample (RLU/ug total protein). For the histochemical staining procedure, the cell monolayers were fixed with 0.5% glutaraldehyde in PBS, and then were incubated with a mixture of 1 mg of 5-bromo-4-chloro-3-indolyl-β-D-galactoside (X-gal) per ml, 5 mM potassium ferrocyanide, 5 mM potassium ferricyanide and 2 mM MgCl₂ in 0.5 ml of PBS. The monolayers were washed with PBS and the blue cells were visualized by light microscopy with a Zeiss ID03 microscope.

Results

Transient Transfection/Infection System.

To rapidly evaluate a panel of potential CAR-binding fiber mutants in the context of viral particles, we have developed a transient transfection/infection system. This system, which is based on pseudotyping a fiberless virus with the mutant fiber proteins, consists of two components. The first is an E1, E3, fiber-deleted adenovirus, Ad5.βgal.ΔF {Von Seggem, Chiu, et al. 1999} (FIG. 1A). This virus, when grown on a non fiber-complementing cell line such as 293T, yields viral particles lacking fiber protein. For purposes here, these fiberless virions are designated Ad5.βgal.ΔF/F⁻. If Ad5.βgal.ΔF is produced on the fiber-complementing cell line 633 {Von Seggem, Huang, et al. 2000}, the virions contain a full complement of wildtype fiber protein on the surface and is referred to as Ad5.βgal.ΔF/F⁺. The second component of the system is an expression plasmid that supplies fiber protein to the assembling virus in trans. This plasmid, pDV60, is designed to express high levels of fiber protein {Von Seggem, Huang, et al. 2000}.

The transient transfection/infection system is shown schematically in FIG. 1B. Transfection of 293T cells by the pDV60-based fiber-expression plasmid results in high levels of fiber production in the cells. Twenty-four hours later, the cells are infected with Ad5.βgal.ΔF/F⁺ that has been previously pseudotyped with wildtype fiber by growth in 633 cells. Approximately three days later, the infected cells are collected and viral particles, now pseudotyped with the fiber protein supplied in trans by the fiber-expression plasmid, are purified. In this way, any plasmid-encoded fiber proteins that are capable of trimerization and incorporating into the viral particles will complement Ad5.βgal.ΔF. Ad5.βgal.ΔF that is pseudotyped either by growth in 633 cells or by transient transfection with a fiber expression plasmid is designated Ad5.βgal.ΔF/F⁺. The function of these modified fiber proteins in the context of a viral particle can then be tested for their ability to mediate fiber-dependent Ad infection and gene transfer.

To compare the level of fiber protein incorporated onto Ad5.βgal.ΔF/F⁺ viral particles generated by this system with the levels in Ad5.βgal.wt, Western immunoblot analysis was performed. Equal particle numbers of Ad5.βgal.ΔF/F⁻, Ad5.βgal.ΔF/F⁺ pseudotyped by pDV60-encoded fiber protein, and Ad5.βgal.wt were evaluated for fiber and penton protein levels. As reported previously {Von Seggem, Chiu, et al. 1999}, the Ad5.βgal.ΔF/F⁻ virions lacked any detectable fiber protein, and Ad5.βgal.wt contained expected levels of the 62kDa fiber protein. Importantly, the level of pDV60-encoded fiber protein incorporated into the Ad5.βgal.ΔF/F⁺ pseudotyped virions using the transient transfection/infection system was equivalent to the level of fiber protein in the Ad5.βgal.wt particles. The equivalent loading of viral particles was demonstrated by detection of the 68 kDa penton monomer for each vector. These results indicate that expression of fiber protein in trans from this expression plasmid can complement Ad5.βgal.ΔF and can result in a level of fiber protein on the capsid that is indistinguishable from that of an adenovirus containing fiber within its genome.

Fiber Mutation Analysis. xThe transient transfection/infection system was then used to evaluate a series of mutations in the fiber knob for their effect on CAR-mediated gene transfer of Ad5 particles. A panel of expression plasmids encoding fourteen mutant fiber proteins was constructed (Table 2). As controls, the wildtype fiber (pDV60) and a null construct (pDV55) were used {Von Seggern, Huang, et al. 2000}. These plasmids were transfected into 293T cells, followed by infection with Ad5.βgal.ΔF/F⁺. The resulting virions obtained from this procedure were thus pseudotyped with the plasmid-encoded fibers. The expression and assembly of each fiber protein into the adenoviral capsid was examined by Western immunoblot analysis of the CsCl-purified virus stocks. The relative levels of fiber protein on the capsid were compared with the amount of penton protein to control for equal loading of viral particles in each lane. The fiber proteins encoded by most mutants were sufficiently expressed, trimerized and incorporated into the Ad5.βgal.ΔF viral particles and the 62 kDa fiber monomer was detected in this analysis. Analysis of the KO11 mutant displayed the expected protein of approximately 48kDa although this truncated fiber protein was not incorporated to the same level as wild-type fiber. These results demonstrate that the mutations introduced into the majority of these fiber genes did not impair their ability to be expressed, trimerized and incorporated into viral particles at levels indistinguishable from wild-type fiber. However, mutants KO2 (SEQ ID NO:6), KO1+2 (SEQ ID NO:8), KO2a (SEQ ID NO:10), and KO11 showed lower levels of incorporated fiber protein although KO11 may have a reduced immunoreactivity with an antiserum that was generated against the full-length wildtype Ad5 fiber protein. Analysis of the relative expression level and trimerization ability of these mutants on non-denaturing polyacrylamide gels showed lower levels of fiber monomer and trimer, indicative of deficiencies in the steady-state levels of these mutant proteins. Except for KO11, which is a deletion of the entire knob, all of these mutants have a mutation at V441 in common.

Having demonstrated efficient expression in trans and virion incorporation of most of these mutant fiber proteins, we next evaluated the affects of these mutations on functional CAR-binding properties. We did this by comparing the transduction efficiency on Hela cells of virions pseudotyped with mutant fiber protein and those pseudotyped with wildtype fiber protein. Transduction efficiency was measured in two ways. A chemiluminescence reporter assay was used to measure the level of adenoviral-encoded β-galactosidase activity and the values (RLU/ug total cellular protein) from one representative experiment are shown in Table 2. A total of five to six separate transductions were performed and the mean β-galactosidase activity values (RLU/ug total cellular protein) were calculated for each adenoviral vector containing the individual fiber mutants. These values were then normalized to the β-galactosidase activity chemiluminescence values obtained with the wildtype fiber to obtain the relative activity of each mutant compared to wildtype fiber. The F−, fiberless vector displayed the most significant, 1000-fold reduction in transduction with only 0.1% of wt activity demonstrating the need for fiber in the efficient transduction of HeLa cells. The KO1 mutation displayed approximately a 70-fold reduction resulting in only 1.4% wt β-galactosidase activity levels. The KO2 (SEQ ID NO:6) mutation resulted in an approximately 167-fold reduction with 0.6% wt activity. The KO2a (SEQ ID NO:10), KO2b (SEQ ID NO:12), and KO2c (SEQ ID NO:14) constructs were designed to identify the amino acid mutation responsible for the significant reduction in CAR interaction. In this comparison, it was revealed that the deletion of amino acid V441 reduced CAR interaction greatest as this single deletion in KO2a (SEQ ID NO:10) resulted in the 167-fold reduction of wt activity and the deletion of K442 had no further effect. The most potent mutation was found with combining the KO1 (SEQ ID NO:4) and KO2 (SEQ ID NO:6) mutations in the KO1+2 (SEQ ID NO:8) construct as this combination resulted in a 1000-fold reduction with only 0.1% wt levels found. A dramatic reduction in transduction efficiency was also found for the KO 11 mutant with a -125-fold reduction of β-galactosidase activity when the entire knob domain was deleted. Significant decreases were also observed for KO3 (SEQ ID NO:16), KO4 (SEQ ID NO: 18), and KO5 (SEQ ID NO:20) although not as dramatic. Little to no effect on fiber-mediated transduction and gene expression was noted for KO4+5, KO8, KO9 and KO10. The average percent wild-type β-galactosidase activity for each pseudotyped adenoviral vector is shown graphically in FIG. 2.

We also analyzed the transduction efficiency of the pseudotyped Ad5.βgal.ΔF/F⁺ by measuring the percentage of cells that were positive for the lacZ reporter gene. This was done by staining the transduced cell monolayers with X-gal. For all mutants, the histochemical data was consistent with the chemiluminescence data. At 1000 particles per cell, Hela cells infected with Ad5.βgal.ΔF/F⁺ pseudotyped with pDV60 showed a high percentage of blue, X-gal positive cells, while Ad5.βgal.ΔF/F⁻ pseudotyped with pDV55 demonstrated very few if any blue cells. The mutants KO1 (SEQ ID NO:4), KO2a (SEQ ID NO:10), and KO2 (SEQ ID NO:6) and KO2c (SEQ ID NO:14), which showed dramatically lower β-galactosidase activity (Table 2 and FIG. 2), also showed extremely low numbers of blue cells as expected. KO4 showed an intermediate reduction in β-galactosidase activity and in the number of X-gal stained positive cells while KO10 had little effect on transduction efficiency by either measure (FIG. 2).

Discussion

One approach to selective cell transduction is to manipulate the fiber protein to redirect the receptor specificity to a particular cell type. There are two requirements for such a targeted adenovirus gene therapy vector. One is ablation of the natural tropism and the second is the introduction of a novel tropism to desired target cell types. We developed a novel system to analyze mutant fiber proteins in the context of the viral particle. Using this system, we identified a number of mutant fiber proteins that retained the ability to incorporate into viral particles but demonstrated a reduction in fiber-mediated gene transfer due to a diminished interaction with CAR. The most dramatic mutations were KO11 (A404-581) and the mutations localized to the fiber AB loop (KO1: S408E, P409A) (SEQ ID NO:4) and the CD loop (KO2: AV441,K442) (SEQ ID NO:6). We generated pseudotyped viral particles containing fiber proteins that incorporates both of these mutations and then directly demonstrated an effect on viral transduction efficiency. In addition, we have identified a novel region of the Ad5 fiber, in the CD loop, that is involved in mediating viral transduction. All mutants that incorporated amino acid changes within this region displayed a reduction in fiber-mediated gene transfer including KO1+2 (SEQ ID NO:8), KO2 (SEQ ID NO:6), KO2a (SEQ ID NO:10), KO1 (SEQ ID NO:4), KO2b (SEQ ID NO:12) and KO2c (SEQ ID NO:14) that resulted in a 1000 to 12-fold reduction. The KO1+2 mutation containing a two amino acid substitution in the A:B loop and a two amino acid deletion in the C:D loop demonstrated the most potent reduction in gene transfer which is greater than either mutation alone. These data suggest that there may be simply a disturbance in overall structure or a cooperative interaction in CAR binding between these two loop regions.

The KO2 (AV441,K442) (SEQ ID NO:6) and KO2a (AV441) (SEQ ID NO:10) mutants showed a significant decrease in transduction efficiency, greater than 160-fold. A portion of this reduction is undoubtedly due to the lower levels of the mutant fiber protein on the viral capsid. However, a significant reduction in transduction efficiency has also been observed for a virus that has the identical KO2 mutation introduced genetically into the viral genome. This virus has a full complement of the mutant fiber protein on the capsid and still shows a dramatic reduction in transduction efficiency in all cell types tested.

We found here that fiber proteins containing amino acid mutations Δ509-510 (KO4) (SEQ ID NO:18) and Δ538-539 (KO5) (SEQ ID NO:20) had reduced capability for transducing Hela cells compared to virus particles pseudotyped with wildtype fiber protein. This suggests that in the Ad5 fiber, these residues on the adjacent monomer are involved in CAR-binding.

The second requirement for an adenovirus that transduces in a cell-type specific manner is the introduction of a novel tropism. The most efficient means is by genetic modification of the fiber gene. Krasnykh et al. {Krasnykh, Dmitriev, et al. 1998} have shown that the HI loop is an appropriate location in the fiber protein to insert peptides with novel receptor specificities. For example, the cRGD ligand (SEQ ID NO:43) {Pasqualini, Koivunen, et al. 1995} inserted into the HI loop has been shown to expand the tropism of Ad both in vitro {Dmitriev, Krasnykh, et al. 1998} and in vivo {Reynolds, Dmitriev, et al. 1999}. One advantage of the transient transfection/infection system described here is that there is no need for a pseudoreceptor system to propagate virions that do not bind CAR. CAR binding is needed for efficient viral production. The production of high titer vector stocks containing ablated fiber-CAR interactions is difficult without an alternative cell-binding pathway. Virus production in the transient transfection/infection system involves a single round of replication that results in a viral capsid pseudotyped with fiber mutants expressed in trans. It should be possible therefore to more easily test combinations of CAR-binding mutations and targeting ligands for their ability to mediate transduction.

TABLE 1
Oligonucleotides used in Ad5 fiber gene mutagenesis.

| | | | |
|---|---|---|---|
| Fiber expression | plasmid | Oligonucleotide sequence | Fiber mutation^{†} |

| | | | |
|---|---|---|---|
| pDKO1 | Seq. ID 21 | 5′-ACCACACCAGCTCCAGAGGCTAACTGTAGACTAAATGC-3′ | S408E, P409A |
| | Seq. ID 22 | 5′-GCATTTAGTCTACAGTTAGCCTCTGGAGCTGGTGTGTT-3′ | |
| pDKO2 | Seq. ID 23 | 5′-ACAGTTTCAGTTTTGGCCGGCAGTTTGGCTCCAATATC-3′ | ΔV441, K442 |
| | Seq. ID 24 | GATATTGGAGCCAAACTGCCGGCCAAAACTGAAACTGT-3′ | |
| pDKO2a | Seq. ID 25 | 5′-ACAGTTTCAGTTTTGGCTAAAGGCAGTTTGGCTCCA-3′ | ΔV441 |
| | Seq. ID 26 | 5′-TGGAGCCAAACTGCCTTTAGCCAAAACTGAAACTGT-3′ | |
| pDKO2b | Seq. ID 27 | 5′-GTTTCAGTTTTGGCTGTTGCTCAGTTTGGCTCCAATA-3′ | ΔK442 |
| | Seq. ID 28 | 5′-TATTGGAGCCAAACTGCCAACAGCCAAAACTGAAAC-3′ | |
| pDKO2c | Seq. ID 29 | 5′-GTTTCAGTTTTGGCTGCTGCAGGCAGTTTGGCTCCA-3′ | V441A, K442A |
| | Seq. ID 30 | 5′-TGGAGCCAAACTGCCTGCAGCAGCCAAAACTGAAAC-3′ | |
| pDKO3 | Seq. ID 31 | 5′-GCTCATCTTATTATAGAATTCGACGAAAATGGAGTG-3′ | R460E |
| | Seq. ID 32 | 5′-CACTCCATTTTTCGTCGAATTTCTATAATAAGATGAGC-3′ | |
| pDKO4 | Seq. ID 33 | 5′-GCTTTATCCAAAATCTCACACTGCCAAAAGTAACATTGTC-3′ | ΔG509, K510 |
| | Seq. ID 34 | 5′-GACAATGTTACTTTTGGCAGTGTGAGATTTATGGATAAGC-3′ | |
| PDKO5 | Seq. ID 35 | 5′-CTAACCATTACACTAAACCAGGAAACAGGAGACAC-3′ | ΔG538, T539 |
| | Seq. ID 36 | 5′-GTGTCTCCTGTTTCCTGGTTTAGTGTAATGGTTAG-3′ | |
| PDKO8 | Seq. ID 37 | 5′-ATAAGATTTTGACGAAACTGGAGTGCTACTAAAC-3′ | N464T |
| | Seq. ID 38 | 5′-GTTTAGTAGCACTCCAGTTTCGTCAAATCTTAT-3′ | |
| PDKO9 | Seq. ID 39 | 5′-TTTGACGAAAATGGACACCTACTAAACAATTTCC-3′ | V466H |
| | Seq. ID 40 | 5′-GGAATTGTTTTAGTAGGTGTCCAGTTTCGTCAAA-3′ | |
| PDKO10 | Seq. ID 41 | 5′-AACCTATCAGCTTATGCAAAATCTCACGGTAAA-3′ | P505A |
| | Seq. ID 42 | 5′-TTTACCGTGAGATTTTGCATAAGCTGATAGGT-3′ | |

⁵⁵⁴ numbering of amino acid residues as in Xia et al 1994.

TABLE 2
Transduction efficiency of pseudotyped Ad5.βgal.ΔF/F⁺
on Hela cells.

| | | | Representative | |
|---|---|---|---|---|
| | | | avg | |
| Fiber | | | □gal activity | Mean |
| expression | Fiber mutant | | (RLU/□g | % wt |
| plasmid | Designation | Mutation^{†} | protein) | (±SD)** |
| pDV60 | Wildtype | None | 529882.0 | 100.0 |
| | (wt) | | | (±0.9) |
| pDV55 | F | Null | 266.3 | 0.1 |
| | | | | (±0.1)* |
| pDKO1 | KO1 | S408E, P409A | 7618.0 | 1.4 |
| | | | | (±0.8)* |
| pDKO1 + 2 | KO1 + 2 | S408E, P409A, | 472.3 | 0.1 |
| | | ΔV441, K442 | | (±0.0)* |
| pDKO2 | KO2 | ΔV441, K442 | 3323.7 | 0.6 |
| | | | | (±0.2)* |
| pDKO2a | KO2a | ΔV441 | 4002.0 | 0.6 |
| | | | | (±0.2)* |
| pDKO2b | KO2b | ΔK442 | 44399.0 | 8.3 |
| | | | | (±0.6)* |
| pDKO2c | KO2c | V441A, | 53336.7 | 8.5 |
| | | K442A | | (±2.2)* |
| pDKO3 | KO3 | R460E | 359229.7 | 63.3 |
| | | | | (±9.1)* |
| pDKO4 | KO4 | ΔG509, K510 | 212987.7 | 38.2 |
| | | | | (±2.8)* |
| pDKO5 | KO5 | ΔG538, T539 | 331349.7 | 58.3 |
| | | | | (±7.1)* |
| pDKO4 + 5 | KO4 + 5 | ΔG509, K510, | 499740.3 | 91.1 |
| | | ΔG538, T539 | | (±12.1) |
| pDKO8 | KO8 | N464T | 469705.7 | 92.6 |
| | | | | (±16.9) |
| pDKO9 | KO9 | V466H | 391442.7 | 80.9 |
| | | | | (±15.1) |
| pDKO10 | KO10 | P505A | 447260.3 | 79.6 |
| | | | | (±6.2) |
| pDKO11 | KO11 | Δ404-581 | 4521.0 | 0.8 |
| | | | | (±0.1)* |

RLU, relative light units. Value represents the average of three wells.
^{†}numbering of fiber amino acid residues as in Xia et al. 1994.
**% wildtype represents the mean (±SD) of the βgal activity of Ad5.βgal.ΔF pseudotyped with each corresponding fiber mutant in 5-6 separate transductions. All values were nonnalized to wildtype (pDV60) at 100%.
*significantly different from wt fiber using an unpaired, two-tailed t-test analysis, p < 0.001

### Example 2

Description of an Av1nBg Virus Containing a Fiber With the cRGD Targeting Moiety and the ΔV441-K442 (KO2) CAR-binding Mutation

Plasmid Description:

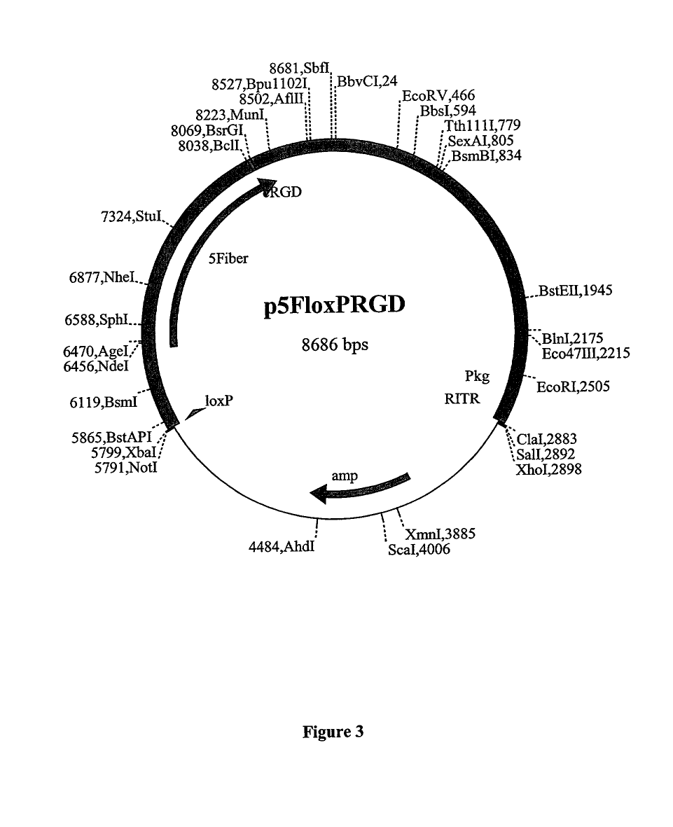

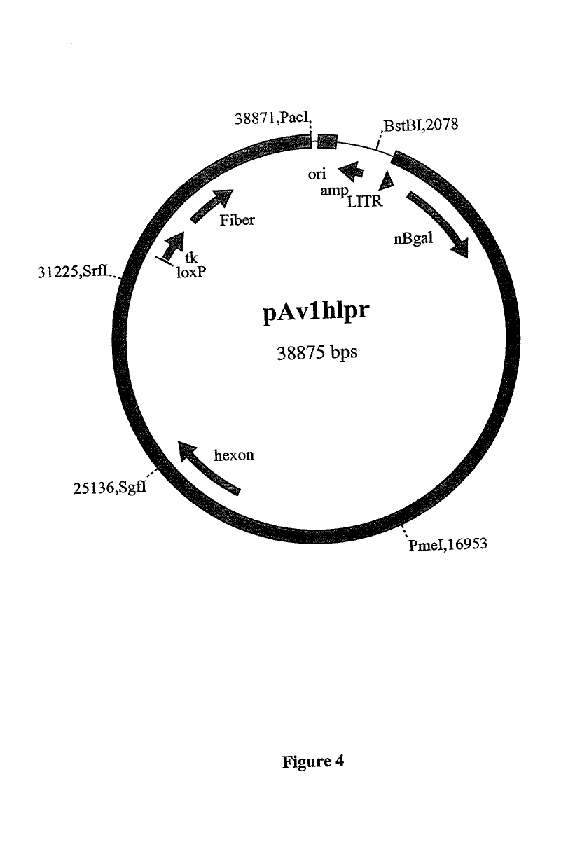

The following three plasmids were used to rescue infectious adenoviruses containing modified fibers. pSF1oxPRGD is a shuttle plasmid used to incorporate modifed fibers into the Ad genome (FIG. 3). It consists of the final 6kb of DNA from the right end of an Av1 genome. A lox site was inserted upstream of the fiber gene. In addition, the Ad5 packaging signal has been inserted near the right inverted terminal repeat (RITR). pAv1hlpr is a helper plasmid that consists of an entire Av1 genome with the exception of the right ITR (FIG. 4). It has a reporter gene inserted in the E1 region that encodes a nuclear-localized β-gal protein, the HSV thymidine kinase gene inserted in the E3 region, and a lox site inserted in a location identical to that in p5FloxPRGD. pCre is a plasmid that constitutively expresses the Cre recombinase. It consists of the Cre gene cloned into the expression plasmid pcDNA1.1zeo⁺ (Invitrogen). None of these plasmids are capable of producing infectious Ad on their own. But when these three plasmids are cotransfected into a complementing cell line, the Cre protein mediates recombination between the lox sites in p5FloxPRGD and pAv1hlpr, reconstituting a full length Av1 genome, which is then capable of producing infectious virus.

Generation of KO2 Fiber Mutations:

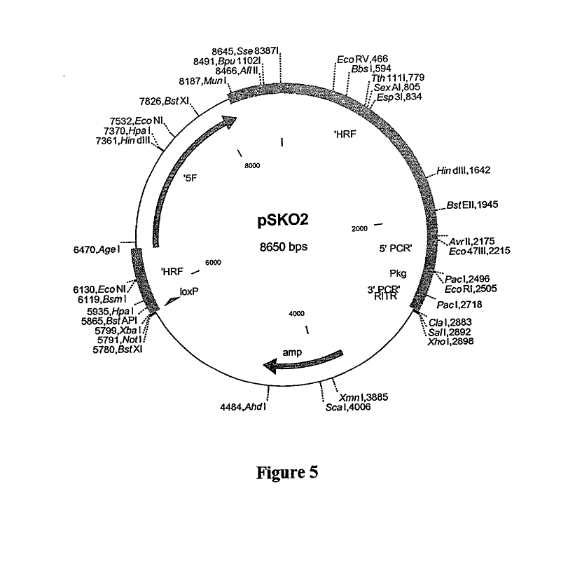

All amino acid changes were incorporated into the fiber gene using the p5FloxPRGD adenoviral shuttle plasmid as the template. This shuttle plasmid encodes a fiber that contains a cRGD peptide sequence, HCDCRGDCFC (SEQ ID NO:43), inserted in the HI loop. The cRGD peptide has been shown to bind to αᵥ-integrins on the cell surface. Amino acid residues V441 and K442 in the CD loop of the fiber gene were deleted using the QuickChange Site-Directed Mutagenesis system (Stratagene, La Jolla Calif.). Deletion of these residues has been shown in the transient transfection/infection system to dramatically inhibit transduction of the Hela cell line which expresses the adenoviral receptor, CAR The resulting shuttle plasmid was called pSKO2 (FIG. 5).

Generation of Adenoviral Vectors with CD Loop Mutations:

The mutagenized fiber gene was incorporated into the adenoviral DNA backbone by cre-lox recombination. To do this, the pSKO2 shuttle plasmid and the pAvlhlpr helper plasmid were cotransfected with pCre into 633 cells, a cell line that expressed wildtype fiber {Von Seggern, Huang, et al. 2000}. Expression of the Cre recombinase from pCre mediates recombination between lox sites in pSKO2 and pAv1hlpr, resulting in full length Av1 viral DNA, with nuclear β-Gal transgene in the E1 region. In the 633 complementing cell line, this viral DNA is capable of being packaged into infectious viral particles containing a mixture of wildtype fiber and mutant fibers. Upon observation of cytopathic effect, the virus was purified by standard CsCl centrifugation procedures. This virus was designated Av1nBgHIRGDKO2(633). In order to obtain viral particles containing only the adenoviral-encoded mutant fiber gene with the ΔV441, K442 mutations (SEQ ID NO:6), this viral preparation was used to infect AE1-2a cells, which do not express fiber {Gorziglia, Kadan, et al. 1996}. Viral particles were purified as above. This virus was designated Av1nBgHIRGDKO2.

Comparable Fiber Incorporation in Av1nBgHIRGDKO2:

To ensure that the levels of the mutant fiber on the Av1nBgHIRGDKO2 viral particles were normal relative to viruses with wildtype fiber levels, Western blot analysis was performed. Equivalent amounts of Av1nBg and Av1nBgHIRGDKO2 were subjected to SDS-PAGE. This gel was transferred to a membrane and incubated with rabbit anti-Ad5 fiber and rabbit anti-Ad5 penton polyclonal antisera. The fiber penton ratio on Av1nBgHIRGDKO2 viral particles is indistinguishable from that of Av1nBg, demonstrating that there was no effect of the fiber mutations on the level of fiber protein assembled on the viral capsid.

Transduction Efficiency of Av1nBgHIRGDKO2:

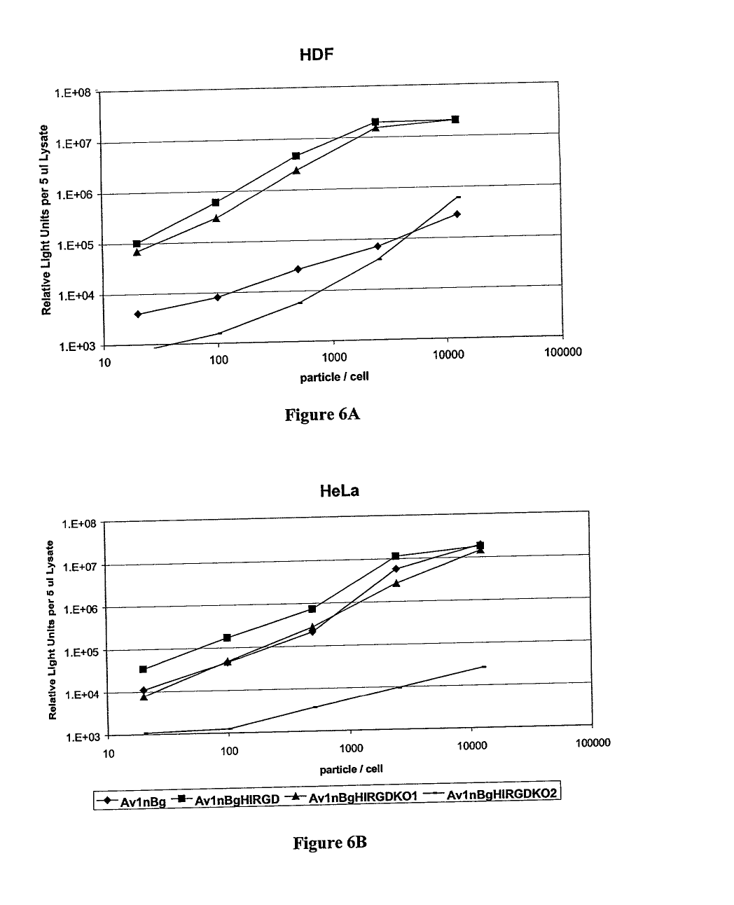

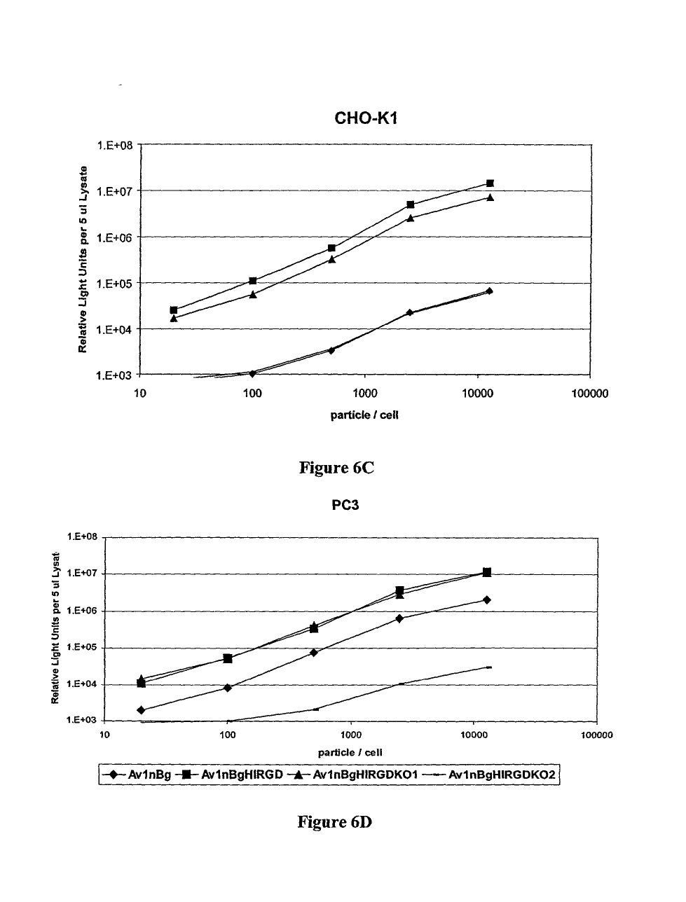

As shown previously in Example 1, adenoviruses pseudotyped with fiber proteins containing deletions of V441 and K442 in the CD loop are severely affected in their ability mediate gene transfer in the CAR-expressing cell line Hela. In order to test the idea that gene transfer by these mutant viruses can be mediated by alternative ligand/receptor interactions, we tested the ability of the Av1nBgHIRGDKO2 virus to transduce HDF, Hela, PC3 and CHO-K1 cell lines (FIG. 6). All of these cells lines express αᵥ integrins on the cell surface and, with the exception of Hela, show poor transduction by adenovirus due to a known or presumed deficiency in CAR levels.

Transduction by Av1nBg, which has wildtype fiber, is extremely inefficient in HDF, PC3 and CHO-KI, as expected (SEQ ID NO:6). Av1nBgHIRGD, which contains RGD in the HI loop, transduces HDF, PC3 and CHO-K1 with much higher efficiency. However, when cRGD is placed in the context of the V441, K442 deletion, as in the Av1nBgHIRGDKO2 virus, transduction efficiency remains inefficient. The levels of Av1nBgHIRGDKO2 transduction in HDF and CHO-K1 cells were similar to Av1nBg and lower in PC3 cells.

This reduction in the inability of Av1nBgHIRGDKO2 to transduce cells through αᵥ-integrins is not due to a defect in the RGD targeting moiety. When Av1nBgHIRGDKO2 is purified from 633 cells, the resulting virus, Av1nBgHIRGDKO2 (633) contains a mixture of the wildtype fiber expressed from the 633 cells, and the mutant fiber expressed from the adenoviral genome. This virus, which has both types of fiber on the virion surface, is now able to mediate efficient transduction of HDF cells. This indicates that the RGD in the fiber is able to mediate transduction of HIDF cells, even in the context of the V441, K442 deletion.

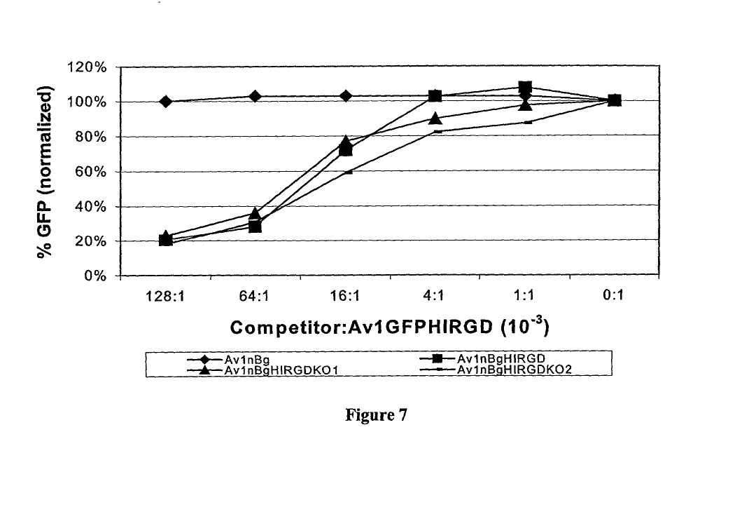

To further demonstrate that the RGD in the fiber containing the V441, K442 deletion is functional, we performed competition experiments (FIG. 7). HDF cells were transduced with Av1GFPHIRGD in the presence of increasing amounts of a competitor virus Av1nBg, Av1nBgHIRGD or Av1nBgHIRGDKO2. The percent of GFP positive cells was then measured. All three competitor viruses successfully inhibited the ability of the Av1GFPHIRGD virus to mediate gene-transfer to comparable degrees.

We conclude from these experiments that the RGD retargeting ligand sequence in a fiber containing the V441, K442 deletion is functional in its ability to mediate binding of αᵥ-integrins (by its ability to inhibit transduction of Av1GFPHIRGD, see FIG. 4) and transduction of cells low in CAR (by its ability to transduce HDF cells when the virus is purified from 633 cells).

In summary, we have described a CD loop mutation, ΔV441 K442 (SEQ ID NO:6), that dramatically reduces the ability of viruses pseudotyped with this fiber protein to mediate gene transfer. Here we show that a viral genome containing the V441 K442 deletion and an RGD insertion in the fiber gene can be rescued. This virus was designated Av1nBgHIRGDKO2. The mutant fiber protein is incorporated into the viral particles at levels indistinguishable from wildtype. The RGD targeting ligand in Av1nBgHIRGDKO2 is functional, as shown in two experiments. First, Av1nBgHIRGDKO2 was able to compete Av1GFPHIRGD transduction of HDF cells which are low in CAR and high in av integrins. Second, we showed that retargeting of Av1nBgHIRGDKO2 through the RGD targeting ligand in HDF cells can be achieved but was dependent on the propagation of the virus in cells expressing wildtype fiber. We conclude that the V441 K442 deletion has dramatically reduced ability to mediate transduction of CAR-expressing cells and that fibers containing these deletions and alternatives targeting ligands are functional.

### Example 3

### Description Of Adenoviral Vectors Containing a Fiber With the S408E,P409A (KO1) CAR-binding Mutation With and Without the cRGD Targeting Moiety

Two recombinant adenoviral vectors were prepared that contain the KO1 fiber mutation and are designated Av3nBgFKO1 and Av1nBgKO1RGD. These vectors contain the KO1 fiber mutation alone or in combination with a cRGD targeting moiety. The construction of these vectors is described below.

Genetic Incorporation of the KO1 Fiber Mutation in Combination with the cRGD Targeting Moiety to Generate Av1nBgKO1RGD.

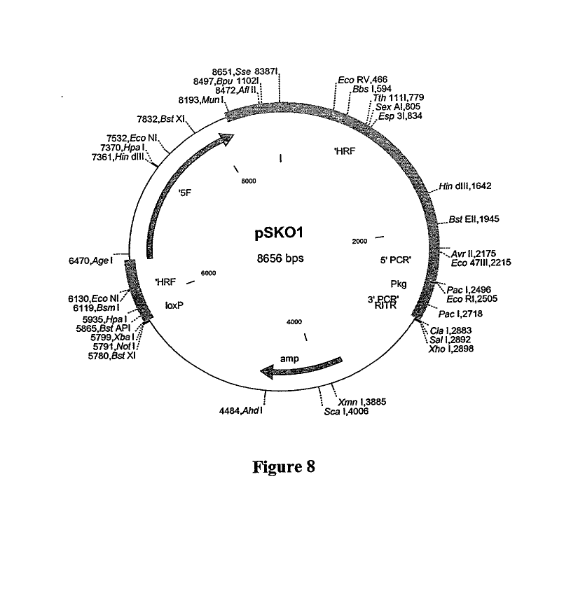

All amino acid changes were incorporated into the fiber gene using the p5FloxPRGD adenoviral shuttle plasmid as the template as previously described in Example 2. This shuttle plasmid encodes a fiber that contains a cRGD peptide sequence, HCDCRGDCFC, inserted in the fiber HI loop. The cRGD peptide has been shown to bind to av-integrins on the cell surface. Amino acid residues 408 and 409 in the AB loop of the fiber gene were changed using the Quickchange site-directed mutagenesis system (Stratagene, La Jolla Calif.). Substitution of these residues has been shown using the transient transfection/infection system to dramatically inhibit transduction of HeLa cells which express the adenoviral receptor, CAR. The resulting shuttle plasmid was called pSKO1 (FIG. 8). The mutagenized fiber gene and the cRGD targeting moiety were incorporated into the adenoviral DNA backbone by cre-lox recombination. To do this, the pSKO1 shuttle plasmid (FIG. 8) and the pAvlhlpr plasmid (FIG. 4) were co-transfected with pCRE (described in Example 2) into 633 cells, a cell line that expresses the wildtype fiber {Von Seggern et al. 2000}. Expression of the CRE recombinase from pCRE mediates recombination between the lox sites in pSKO1 and in pAvlhlpr, resulting in full length Av1 viral DNA with the nuclear β-gal transgene in the E1 region to generate Av1nBgKO1RGD. This virus was initially propagated on 633 cells. After growth on these cells, the virus capsid contained both wildtype and mutant fiber proteins. To obtain viral particles containing only the modified fiber with the KO1 mutation and the RGD moiety, the viral preparation was used to infect AE1-2a cells {Gorziglia, Kadan, et al., 1996}, which do not express fiber.

Genetic Incorporation of the KO1 Fiber Mutation into the Adenoviral Genome.

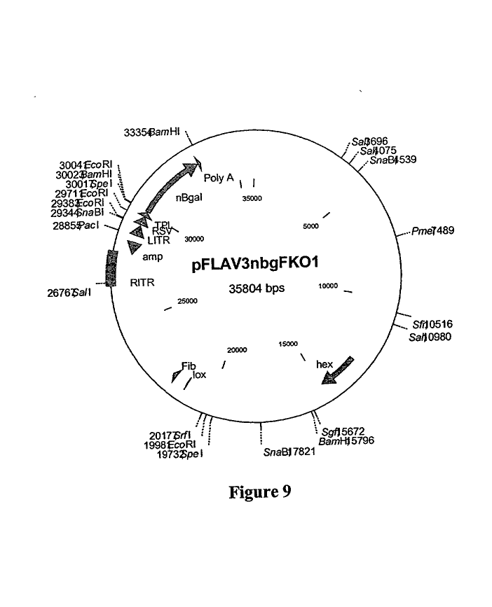

The KO1 mutation alone was incorporated genetically into the adenoviral genome to generate Av3nBgFKO1. The KO1 mutation was cloned into a plasmid containing the full-length Av3 adenoviral genome {Gorzigliz, Kadan, et al., 1996} to generate pFLAv3nBgFKO1 (FIG. 9). Transfections were carried out in 633 cells, and in this fiber complementing cell line, the resulting viral DNA containing the KO1 mutation is capable of being packaged into infectious viral particles containing a mixture of wildtype fiber and mutant fiber proteins. Upon observation of CPE, the virus was purified by standard CsCl centrifugation procedures. In order to obtain viral particles containing only the adenoviral encoded mutated KO1 fiber protein with the S408E, P409A mutations, this viral preparation was used to infect AE1-2a cells, which do not express fiber. Viral particles were purified as described above.

In Vitro Evaluation of Adenoviral Vectors Containing the KO1 Fiber Mutation.

Several recombinant adenoviral vectors were used in these studies to demonstrate the function and specificity of the KO1 fiber mutation and the cRGD targeting moiety. These vectors are described in Table 3.

TABLE 3
Description of recombinant adenoviral vectors used

| Vector | Description |
|---|---|
| Av3nBg | An E1, E2a, E3-deleted adenoviral vector encoding |
| | a nuclear localizing β-galactosidase |
| Av3nBgFKO1 | The same as Av3nBg but containing the KO1 |
| | mutation in the fiber gene |
| Av15FHIRGD | An E1, E3-deleted vector encoding a nuclear |
| | localizing β-galactosidase and containing a cRGD |
| | ligand in the HI loop of fiber |
| Av1nBgKO1RGD | An E1, E3-deleted vector encoding a nuclear |
| | localizing β-galactosidase and containing both |
| | the KO1 fiber mutation and a cRGD ligand in the |
| | HI loop. |

Transduction Efficiency of Adenoviral Vectors Containing AB Loop Mutations.

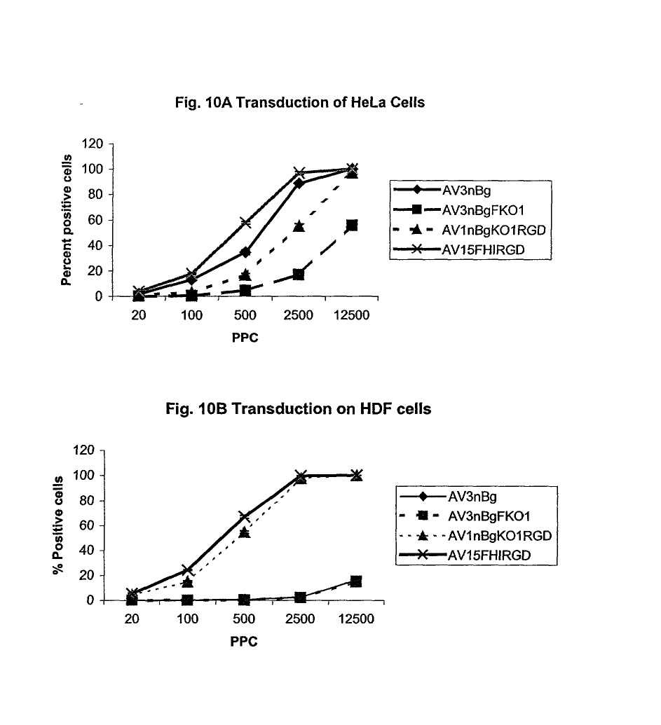

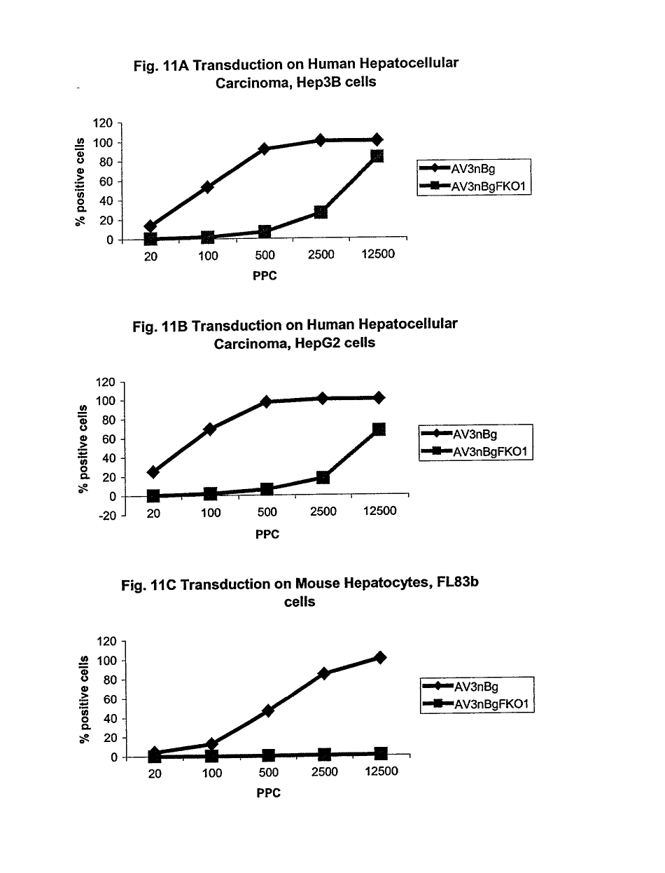

As shown previously in example 1, adenoviruses pseudotyped with fiber proteins containing the S408E, P409A substitutions in the AB loop are severely affected in their ability to mediate gene transfer in HeLa cells, a CAR-expressing cell line. In order to demonstrate the function and specificity of the KO1 mutation and the ability to restore efficient gene transfer by using alternative ligand/receptor interactions, we tested the ability of the KO1-containing recombinant vectors to transduce various cell types. The four vectors listed in Table 3 were compared for transduction efficiency on HeLa cells, human diploid fibroblasts (HDFs), two different human hepatocellular carcinoma cell lines, Hep3Bs and HepG2s, and a mouse hepatocyte cell line, FL83b. The cells were seeded into the wells of a 24-well dish at 1-2×10⁵ cells per well. The next day, the exact number of cells per well was determined for each cell line by counting a representative well for each cell type. The cells were transduced with various numbers of particles per cell (PPC), in triplicate, using each of the four vectors. Twenty-four hours after transduction, the cells were stained with X-gal and the percentage of β-galactosidase expressing cells was determined by counting cells under the microscope. Blue cells were counted in six different fields for each well. The total number of cells per field was determined by counting all cells in three fields from only one well, assuming that the total number of cells per field was the same for a given cell type across all wells. The results showed that transduction of HeLa, Hep3B, HepG2, and FL83b cells by Av3nBgFKO1 was dramatically reduced compared to Av3nBg (FIGS. 10 and 11). HDFs, which express little or no CAR on their surface, were poorly transduced by both Av3nBg and Av3nBgFKO1 (FIG. 10B). As expected, efficient transduction of HDFs was observed using the two vectors containing a cRGD ligand in the HI loop, Av15FHIRGD and Av1nBgKO1RGD (FIG. 10B).

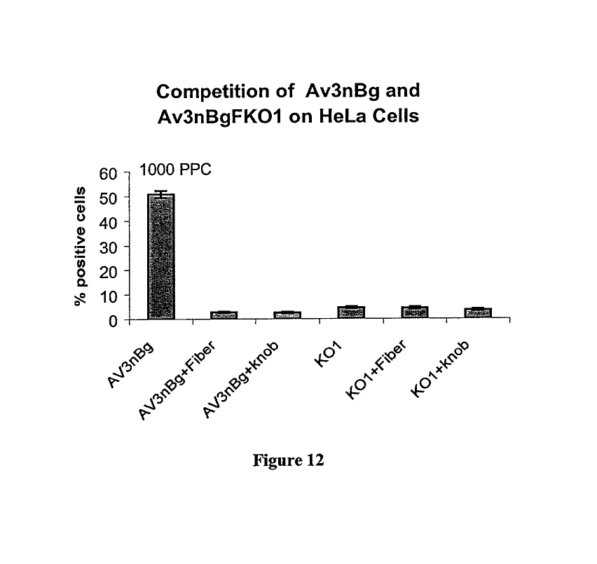

To determine whether the transduction observed in the above study was mediated by fiber-CAR interaction, competition binding experiments using HeLa cells were carried out using either full-length fiber protein or fiber knob as protein competitors (FIG. 12). Competition was carried out by adding 40

g/ml of the full-length fiber or 16

g/ml of the fiber knob directly to the cells for 5 minutes at room temperature prior to infecting with vector. The results showed that either full-length fiber or fiber knob could efficiently block transduction of cells by Av3nBg. However, the low level of transduction of cells by Av3nBgFKO1 was not blocked by full-length fiber or fiber knob. This result shows that the low level of transduction by Av3nBgFKO1 was not mediated by fiber-CAR interaction. Similar results were also obtained on human hepatocytes. Fiber competition of Av3nBgFKO1 on mouse hepatocytes was not evaluated since transduction in the absence of competition was barely detectable.

In Vivo Analysis of Adenoviral Vectors Containing the KO1 Fiber Mutation.

Efficient in vivo targeting of adenoviral vectors requires both ablation of the normal tropism and the addition of a new tropism. We generated adenoviral vectors which were designed to achieve both of those requirements. (See Table 3.) To ablate the normal tropism, the adenovirus fiber protein was genetically modified to knockout its interaction with the coxsackie-adenovirus receptor (CAR). To retarget the vector to a different receptor, a cRGD targeting moiety was inserted into the HI loop of the fiber knob. In vitro studies demonstrate that adenoviral vectors containing mutations in the fiber knob which knockout its ability to interact with CAR are unable to transduce target cells. One particular fiber knockout mutant, called KO1, reduces transduction of HeLa cells to 1.4% of the transduction level observed using a vector containing a wild-type fiber. Furthermore, in vitro data has been generated showing that cRGD ligand insertion into the HI loop of the fiber KO1 mutant restores transduction to levels equivalent to that seen with “wild-type” vector. These vectors were next analyzed in vivo using C57BL/6 male mice to assess the extent of liver detargeting and to determine the tissue distribution of such vectors following tail vein administration and to determine whether insertion of the-cRGD targeting ligand into the HI loop restores liver transduction.

In Vivo Study Design.

The study included 5 cohorts of 5 mice each. Adenoviral vectors encoding nuclear targeted β-galactosidase (nBg) were administered by tail vein injection. The dose was 1×10¹³ particles per kilogram. Mice were sacrificed 3 days after vector administration. Tissues, including liver, lung, heart, kidney, and spleen, were collected. Several assays were utilized to assess the efficiency of liver transduction and the vector biodistribution and included hexon PCR analysis, β-gal immunohistochemistry, and the β-gal Tropix assay. One group of mice received Hanks Balanced Salt Solution (HBSS) instead of adenoviral vector and served as a negative control. A second cohort received Av3nBg, which contains a “wild-type” fiber protein and served as a positive control. A third group received Av3nBgFKO1, a fourth group received Av1nBgKO1HIcRGD, and a fifth group received Av1nBgHIcRGD.

Vector Preparation.

Each vector was diluted into sterile HBSS, at a final concentration of 1×10¹² particles/ml. Mice in-groups 2 through 6 were injected with a volume of 10 ml/kg to achieve a vector dose of 1×10¹³, particles per kg. The HBSS control group received an equivalent dose volume.

Technical Methods.

Administration of control and test articles was by bolus tail vein injection. Dose was determined from body weights obtained the day of administration. Animals were sacrificed approximately 72 hours after vector administration by carbon dioxide asphyxiation. Liver, heart, lung, spleen, and kidney were collected from each animal. Slices of each tissue approximately 2-3 mm thick were placed in neutral buffered formalin to preserve the sample for β-galactosidase immunohistochemistry. For optimal histology, one piece of each liver lobe, a lobe of the lung, a whole kidney, an end cross sectional piece of the spleen, and half of the heart cut longitudinally, were placed into the same container of neutral buffered formalin. The remaining tissue from each organ was placed into a 1 ml cryovial and frozen in dry ice to preserve it for hexon PCR analysis to determine vector content. For the liver, pieces of each lobe were frozen in dry ice to preserve it for hexon PCR analysis and other pieces of each lobe were placed in a “Tropix” vial, and frozen on dry ice.

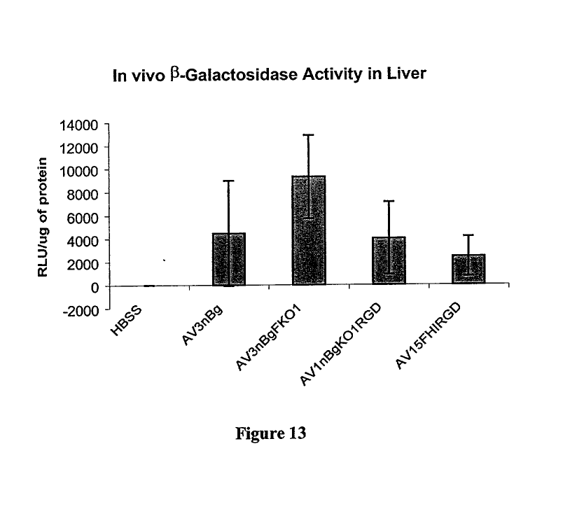

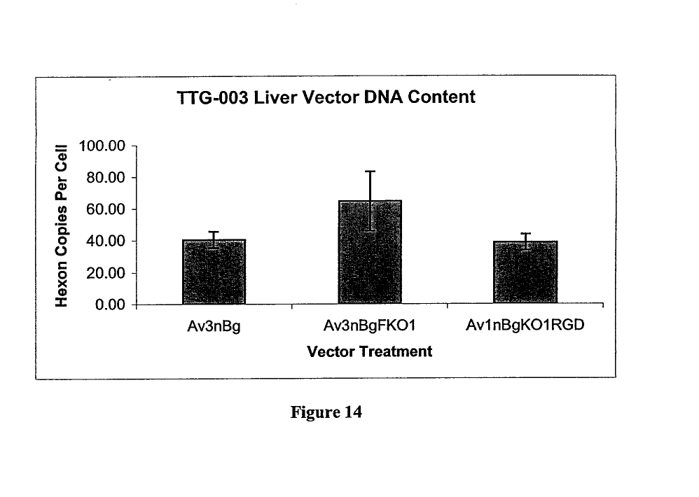

The results of the immunohistochemical staining for β-galactosidase expression showed that Av3nBg, Av3nBgFKO1, Av15FHIRGD, and Av1nBgKO1RGD all yielded efficient transduction of hepatocytes. Av3nBgFKO1 yielded a higher percentage of β-galactosidase expressing cells and a more intense staining than Av3nBg. This result was completely unexpected since Av3nBgFKO1 transduction of various cells in culture was dramatically reduced. Evaluation of β-galactosidase expression in mouse livers by a chemiluminescent assay (FIG. 13) confirmed the results of the immunohistochemical staining. Mice that received Av3nBgFKO1 demonstrated higher levels of expression than those that received Av3nBg. A measurement of the vector content in hepatocytes was determined by a semi-quantitative hexon PCR assay (FIG. 14). The results were consistent with both the immunohistochemical staining and the chemiluminescent assay. The vector content in hepatocytes was approximately 35% higher in the mice that received Av3nBgFKO1 than in those that received Av3nBg.

Summary.

The fiber AB loop mutation contained in Av3nBgFKO1 ablates interaction with human and mouse CAR in vitro. However, in vivo this fiber AB loop mutation behaves quite unexpectantly as it was found to dramatically enhance adenoviral-mediated gene transfer to liver and other organs and results in increasing vector potency. This fiber modification will be useful for in vivo gene therapy strategies and will allow for lower doses of adenoviral vectors to be used systemically.

### Example 4

### Increased Liver Transduction Using Av3nBgFKO1 in Three Different Mouse Strains

The following experiment was done to determine whether the increased liver transduction observed with Av3nBgFKO1 compared to that using Av3nBg could be reproduced in various mouse strains.

In Vivo Analysis of Adenoviral Vectors Containing the KO1 Fiber Mutation.

C57BL/6, Balbc, and CD1 male mice were purchased from Harlan Sprague Dawley (Indianapolis, Ind.). When the mice were 5 weeks of age they received either HBSS (vehicle control), Av3nBg, or Av3nBgFKO1 via tail vein injection at a dose of 1×1013 particles per kg which is approximately 2×1011 particles per mouse. Cohorts of five mice received each treatment. The vector was diluted to 1×10¹² particles per ml using Hanks Balanced Salt Solution (HBSS) immediately prior to injection. Three days after vector delivery, the animals were sacrificed and tissues including liver, lung, heart, kidney, and spleen were collected.

Technical Methods for Galacto-Light Plus™ Chemiluminescent Assay.

These tissue samples were used to analyze β-galactosidase expression using the Galacto-Light Plus™ chemiluminescent assay (Tropix, Inc., Foster City, Calif.) systems. Tissue samples were collected in lysis matrix tubes containing two ceramic spheres (Bio101, Carlsbad, Calif.) and frozen on dry ice. The tissues were thawed and 500 μl of lysis buffer from the Galacto-Light Plus kit was added to each tube. The tissue was homogenized for 30 seconds using a FastPrep System (Bio101, Carlsbad, Calif.). Liver samples were homogenized for an additional 30 seconds. The β-galactosidase activity in tissue lysates was assayed according to the manufacturer's instructions.

Results.

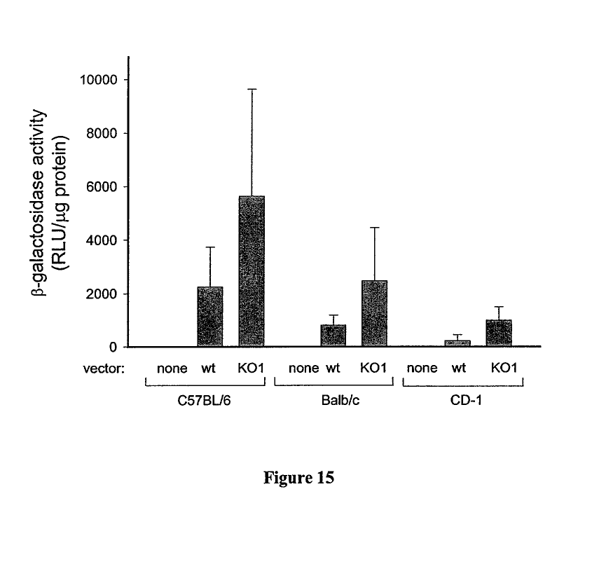

The results (FIG. 15) showed that, on average, Av3nBgFKO1 yielded higher levels of liver transduction than Av3nBg in all three mouse strains.

Technical Methods for β-Galactosidase Immunohistochemistry.

In addition, slices of each tissue approximately 2-3 mm thick were placed in 10% neutral buffered formalin. After fixation, these samples were embedded in paraffin, sectioned, and analyzed by immunohistochemistry for β-galactosidase expression. A 1:1200 dilution of a rabbit anti-β-galactosidase antibody (ICN Pharmaceuticals, Inc.; Costa Mesa, Calif.) was used in conjunction with a Vectastain ABC kit from Vector Laboratories, Inc. (Burlingame, Calif.) to visualize positive cells.

Results.

The results showed that Av3nBgFKO1 yielded higher levels of hepatocyte transduction than Av3nBg in all three mouse strains.

### Example 5

### Av3nBgFKO1 Transduced Primary Mouse Hepatocytes in Culture Relatively Poorly Compared To Av3nBg

The transduction efficiencies of Av3nBg and Av3nBgFKO1 were evaluated on primary mouse hepatocytes.

Isolation and Culturing of Primary Murine Hepatocytes.

Primary murine hepatocytes were isolated from adult, male CD-1 mice following a two-step collagenase perfusion via the portal vein, modified from published procedures (Seglen, Methods Cell Biol 1973, 13:29-83; Liddle et al., J Gastro Hepatol 1998, 13:855-858; Marc et al., Eur J Biochem 2000, 267:963-970). The liver was perfused in situ with Liver Perfusion Medium (Life Technologies, Gaithersburg, Md.) followed by treatment with Liver Digest Medium (collagenase-dispase; Life Technologies). The liver was minced, and cells were washed and centrifuged three times in Hepatocyte Wash medium (enriched William's E; Life Technologies) before being resuspended in Hepatocye Attachment Medium (Modified William's E, supplemented with 1% pen-strep and 5% FBS; Life Technologies). Viability was assessed by trypan blue exclusion. Cells were plated at approximately 1×10⁵ viable cells per well on collagen type I-coated 24-well plates and allowed to attach for 2 hr at 37° C. in 5% C02. After 2 hr, unattached cells and media were removed, cells were washed and cultured in HepatoZYME-SFM (Life Technologies). Immunohistochemical staining for albumin confirmed the identity of these cells as hepatocytes.

Transduction Efficiency of Av3nBgFKO1 on Primary Murine Hepatocytes.

Approximately 24 hours after plating, the cells were transduced with the adenoviral vectors Av3nBg and Av3nBgFKO1 at various numbers of particles per cell, ranging from 0 to 12,500. The cells were incubated with adenoviral vector for 1 hour at 37° C. in a total volume of 0.2 ml of culture medium. Next, the cell monolayers were washed once with PBS, then 1 ml of the appropriate culture medium was added to each well. The cells were incubated for 24 hours to allow for β-galactosidase expression. The cell monolayers were then fixed and stained with X-Gal for 24 hours. The percentage of transduction was determined by light microscopy by counting the number of transduced, blue cells per total cells in a high-power field with a Nikon CK1 microscope; three fields were counted per well. Each vector dose was carried out in triplicate and the average percentage of transduction per high-power field (n=3 wells) was determined. The mean percent transduction obtained from at least three independent experiments was determined.

Results.

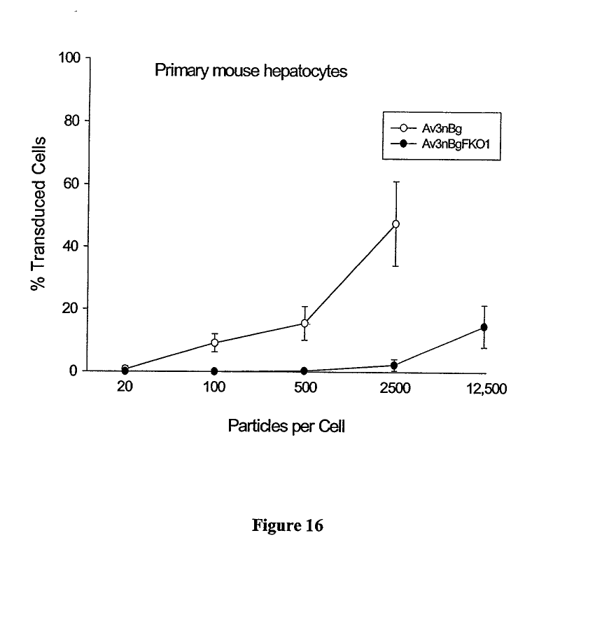

The results (FIG. 16) showed dramatically reduced transduction of primary mouse hepatocytes using Av3nBgFKO1 compared to Av3nBg.

### REFERENCES

Dmitriev, I., V. Krasnykh, C. R. Miller, M. Wang, E. Kashentseva, G. Mikheeva, N. Belousova, and D. T. Curiel. 1998. An adenovirus vector with genetically modified fibers demonstrates expanded tropism via utilization of a coxsackievirus and adenovirus receptor-independent cell entry mechanism. J Virol 72:9706-9713.

Gorziglia, M. I., M. J. Kadan, S. Yei, J. Lim, G. M. Lee, R. Luthra, and B. C. Trapnell. 1996. Elimination of both E1 and E2 from adenovirus vectors further improves prospects for in vivo human gene therapy. J Virol 70:4173-4178.

Harbury, P. B., T. Zhang, P. S. Kim, and T. Alber. 1993. A switch between two-, three-, and four-stranded coiled coils in GCN4 leucine zipper mutants. Science 262:1401-1407.

Horton, R. M., Z. L. Cai, S. N. Ho, and L. R. Pease. 1990. Gene splicing by overlap extension: tailor-made genes using the polymerase chain reaction. Biotechniques 8:528-535.

Krasnykh, V., I. Dmitriev, G. Mikheeva, C. R. Miller, N. Belousova, and D. T. Curiel. 1998. Characterization of an adenovirus vector containing a heterologous peptide epitope in the HI loop of the fiber knob. J Virol 72:1844-1852.

Krasnykh, V. N., G. V. Mikheeva, J. T. Douglas, and D. T. Curiel. 1996. Generation of recombinant adenovirus vectors with modified fibers for altering viral tropism. JVirol 70:6839-6846.

Michael, S. I., J. S. Hong, D. T. Curiel, and J. A. Engler. 1995. Addition of a short peptide ligand to the adenovirus fiber protein. Gene Ther 2:660-668.

Mittereder, N., K. L. March, and B. C. Trapnell. 1996. Evaluation of the concentration and bioactivity of adenovirus vectors for gene therapy. J Virol 70:7498-7509.

Pasqualini, R., E. Koivunen, and E. Ruoslahti. 1995. A peptide isolated from phage display libraries is a structural and functional mimic of an RGD-binding site on integrins. J Cell Biol 130:1189-1196.

Reynolds, P., I. Dmitriev, and D. Curiel. 1999. Insertion of an RGD motif into the HI loop of adenovirus fiber protein alters the distribution of transgene expression of the systemically administered vector. Gene Ther 6:1336-1339.

Stevenson, S. C., M. Rollence, J. Marshall-Neff, and A. McClelland. 1997. Selective targeting of human cells by a chimeric adenovirus vector containing a modified fiber protein. J Virol 71:4782-4790.

Stevenson, S. C., M. Rollence, B. White, L. Weaver, and A. McClelland. 1995. Human adenovirus serotypes 3 and 5 bind to two different cellular receptors via the fiber head domain. J Virol 69:2850-2857.

Von Seggem, D. J., C. Y. Chiu, S. K. Fleck, P. L. Stewart, and G. R. Nemerow. 1999. A helper-independent adenovirus vector with E1, E3, and fiber deleted: structure and infectivity of fiberless particles. J Virol 73:1601-1608.

Von Seggem, D. J., S. Huang, S. K. Fleck, S. C. Stevenson, and G. R. Nemerow. 2000. Adenovirus Vector Pseudotyping in Fiber-Expressing Cell Lines: Improved Transduction of Epstein-Barr Virus-Transformed B Cells. J Virol 74:354-362.

Wickham, T. J., R. R. Granados, H. A. Wood, D. A. Hammer, and M. L. Shuler. 1990. General analysis of receptor-mediated viral attachment to cell surfaces. Biophys J 58:1501-1516.

Wickham, T. J., D. M. Segal, P. W. Roelvink, M. E. Carrion, A. Lizonova, G. M. Lee, and I. Kovesdi. 1996. Targeted adenovirus gene transfer to endothelial and smooth muscle cells by using bispecific antibodies. J Virol 70:6831-6838.

Wickham, T. J., E. Tzeng, L. L. Shears, P. W. Roelvink, Y. Li, G. M. Lee, D. E. Brough, A. Lizonova, and I. Kovesdi. 1997. Increased in vitro and in vivo gene transfer by adenovirus vectors containing chimeric fiber proteins. J Virol 71:8221-8229.

The disclosures of all patents, patent applications, publications (including published patent applications), depository accession numbers, and database accession numbers referred to in this specification are specifically incorporated herein by reference in their entirety to the same extent as if each such individual patent, patent application, publication, depository accession number, and database accession number were specifically and individually indicated to be incorporated in its entirety.

1. A mutated adenoviral fiber protein wherein at least one amino acid in the CD loop of a wild-type fiber protein of an adenovirus from subgroup C, D, or E, or the long wild-type fiber of an adenovirus from subgroup F, has been mutated to reduce or substantially eliminate the ability of said fiber protein to bind to the coxsackievirus-adenovirus receptor (CAR).

2. The mutated adenoviral fiber protein of claim 1, wherein said mutation substantially eliminates the ability of said protein to bind to said CAR.

3. The mutated adenoviral fiber protein of claim 2, wherein said fiber protein is an adenovirus serotype 5 fiber protein.

4. The mutated adenoviral fiber protein of claim 3, wherein said fiber protein contains at least one mutation at amino acid positions 441 and 442 of the wild-type fiber protein.

5. The mutated adenoviral fiber protein of claim 4, wherein said fiber protein further comprises a mutation at one or more of the following amino acid positions of the wild-type fiber protein: 408, 409, 460, 509, 510, 538, and 539.

6. The mutated adenoviral fiber protein of claim 4, wherein said fiber protein further comprises at least one mutation at amino acid positions 408 and 409 of the wild-type fiber protein.

7. A mutated adenovirus serotype 5 fiber protein wherein said fiber protein comprises a mutation at one or more of the following amino acid positions of the wild-type fiber protein: 460, 509, 510, 538, and 539, wherein said mutation reduces or substantially eliminates the ability of said fiber protein to bind to CAR.

8. A polynucleotide encoding the protein of claim 1.

9. A polynucleotide encoding the protein of claim 3.

10. A polynucleotide encoding the protein of claim 4.

11. A polynucleotide encoding the protein of claim 5.

12. A polynucleotide encoding the protein of claim 7.

13. An adenoviral particle comprising the fiber protein of claim 1.

14. An adenoviral particle comprising the fiber protein of claim 3.

15. An adenoviral particle comprising the fiber protein of claim 4.

16. An adenoviral particle comprising the fiber protein of claim 5.

17. An adenoviral particle comprising the fiber protein of claim 7.

18. The adenoviral particle of claim 13 further comprising a targeting ligand included in a capsid protein of said particle.

19. The adenoviral particles of claim 18 wherein said capsid protein is the mutated adenoviral fiber protein.

20. The adenoviral particle of claim 19 further comprising at least one heterologous polynucleotide.

21. The adenoviral particle of any one of claims 14-17 further comprising a targeting ligand included in a capsid protein of said particle.

22. The adenoviral particle of claim 21 wherein said capsid protein is the mutated adenoviral fiber protein.

23. The adenoviral particle of claim 22 further comprising at least one heterologous polynucleotide.

24. An adenovirus packaging cell comprising the polynucleotide of claim 8.

25. A method of making the adenoviral particle of claim 13, comprising the steps of:
transferring the adenovirus genome to be packaged in said particle into the packaging cell of claim 24;
culturing said packaging cell; and
recovering an adenoviral particle produced by said cell.

26. A method of making the adenoviral particle of claim 18 comprising the steps of:
transferring the adenovirus genome to be packaged in said particle into a cell
having adenovirus polynucleotides that provide proteins necessary for the replication,
maturation, and packaging of said genome;
culturing said cell under conditions permitting the production of said particle; and
recovering an adenoviral particle produced by said cell.

27. A method of expressing a heterologous polynucleotide in a cell comprising infecting said cell with the adenoviral particle of claim 20.

28. The method of claim 27, wherein said cell is a mammalian cell.

29. The method of claim 28, wherein said mammalian cell is a primate cell.

30. The method of claim 29, wherein said primate cell is a human cell.

31. A composition comprising the adenoviral particle of claim 18 in a pharmaceutically acceptable carrier.

32. A composition comprising the adenoviral particle of claim 20 in a pharmaceutically acceptable carrier.

33. A mutated adenovirus serotype 5 fiber protein wherein said fiber protein contains mutations at amino acid positions 408 and 409 of the wild-type fiber protein.

34. The mutated fiber protein of claim 33, wherein said protein contains deletions at amino acid positions 408 and 409 of the wild-type fiber protein.

35. The mutated fiber protein of claim 33, wherein said protein contains amino acid substitutions at amino acid positions 408 and 409 of the wild-type fiber protein.

36. The mutated fiber protein of claim 35, wherein glutamic acid is substituted for serine at position 408 and alanine is substituted for proline at position 409 (SEQ ID NO: 4).

37. A polynucleotide encoding the protein of claims 33-36.

38. An adenoviral particle comprising the fiber protein of claims 33-36.

39. The adenoviral particle of claim 38, further comprising a targeting ligand included in a capsid protein of said particle.

40. The adenoviral particle of claim 39, further comprising at least one heterologous polynucleotide.

41. The adenoviral particle of claim 38, wherein at least one of the penton proteins of said particle has been modified to delete the RGD sequence.

42. An adenovirus packaging cell comprising the polynucleotide of claim 37.

43. A method of making the adenoviral particle of claim 38, comprising the steps of:
transferring the adenovirus genome to be packaged in said particle into the packaging cell of claim 42;
culturing said packaging cell; and
recovering an adenoviral particle produced by said cell.

44. A method of making the adenoviral particle of claim 38, comprising the steps of:
transferring the adenovirus genome to be packaged in said particle into a cell having adenovirus polynucleotides that provide proteins necessary for the replication,
maturation, and packaging of said genome;
culturing said cell under conditions permitting the production of said particle; and
recovering an adenoviral particle produced by said cell.

45. A method of expressing a heterologous polynucleotide in a cell comprising infecting said cell with the adenoviral particle of claim 40.

46. The method of claim 45, wherein said cell is a mammalian cell.

47. The method of claim 45, wherein said cell is a primate cell.

48. The method of claim 45, wherein said cell is a human cell.

49. A composition comprising the adenoviral particle of claim 40 in a pharmaceutically acceptable carrier.

50. A method of enhancing adenoviral-mediated gene transfer to and expression in hepatocyte& comprising the steps of administering adenoviral particles of claim 40 to said hepatocytes.

51. A method of enhancing adenoviral-mediated gene transfer to and expression in hepatocytes comprising the steps of:
preparing an adenovirus particle comprising a mutated adenovirus serotype 5 fiber protein, wherein glutamic acid is substituted for serine at amino acid position 408 and alanine is substituted for proline at amino acid position 409, and further comprising a heterologous gene; and infecting hepatocytes with said adenovirus particle.

52. A method of expressing a protein in a mammal comprising the step of administering the adenoviral particle of claim 20 or claim 40 to said mammal, wherein said particle transduces a cell in said mammal and said heterologous polynucleotide expresses said protein in said cell.

53. The method of claim 52, wherein said mammal is a primate.

54. The method of claim 53, wherein said primate is a human.

55. A method of expressing a protein in the liver of a mammal comprising administering a sufficient amount of the adenoviral particles of claim 40 for said particles to transduce cells in the liver of said mammal.

56. The method of claim 55, wherein said amount comprises approximately 1 particle per kilogram of body weight to approximately 10¹³ particles per kilogram of body weight.

57. The method of claim 55, wherein said amount comprises approximately 10⁴ particles per kilogram of body weight to approximately 10¹² particles per kilogram of body weight.

58. The method of claim 55, wherein said amount comprises approximately 10⁸ particles per kilogram of body weight to approximately 10¹¹ particles per kilogram of body weight.

59. An adenoviral vector comprising the polynucleotide of any one of claims 8-12.

60. An adenoviral vector comprising the polynucleotide of claim 37.

61. The adenoviral particle of claims 13, 18, 20, 38, 39, 40, or 41, wherein said adenoviral particle is a replication conditional adenovirus.

62. The adenoviral particle of claim 61, wherein said adenovirus is an oncolytic adenovirus.


SEQUENCE LISTING (43 sequences)

SEQ ID NO: 1 - DNA, 1746 bp, Human adenovirus type 5
  CDS (1)..(1746)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 2 - protein, 581 aa, Human adenovirus type 5
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 3 - DNA, 1746 bp, Artificial Sequence
  Codes for a mutated Human Adenovirus type 5 fiber protein.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccagaggctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 4 - protein, 581 aa, Artificial Sequence
  Codes for a mutated Human Adenovirus type 5 fiber protein.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPEANCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 5 - DNA, 1740 bp, Artificial Sequence
  Codes for a mutated Human Adenovirus type 5 fiber protein. Nucleotides corresponding to amino acids 441(V) & 442(K) of wild-type fiber were deleted.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggccggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 6 - protein, 579 aa, Artificial Sequence
  Codes for a mutated Human Adenovirus type 5 fiber protein. Nucleotides corresponding to amino acids 441(V) & 442(K) of wild-type fiber were deleted.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 7 - DNA, 1740 bp, Artificial Sequence
  Codes for a mutated Human Adenovirus type 5 fiber protein. Combination of the mutant fiber encoded in Seq ID #3 & 5.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccagaggctaactgtagactaaatgcagagaaagatgctaaactcactttggtcttaacaaaatgtggcagtcaaatacttgctacagtttcagttttggccggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 8 - protein, 579 aa, Artificial Sequence
  Codes for a mutated Human Adenovirus type 5 fiber protein. Combination of the mutant fiber encoded in Seq ID #3 & 5.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPEANCRLNAEKDAKLTLVLTKCGSQILATVSVLAGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 9 - DNA, 1743 bp, Artificial Sequence
  Codes for a mutated Human Adenovirus type 5 fiber protein. Nucleotides corresponding to amino acid 441(V) of wild-type fiber was deleted.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 10 - protein, 580 aa, Artificial Sequence
  Codes for a mutated Human Adenovirus type 5 fiber protein. Nucleotides corresponding to amino acid 441(V) of wild-type fiber was deleted.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 11 - DNA, 1743 bp, Artificial Sequence
  Codes for a mutated Human Adenovirus type 5 fiber protein. Nucleotides corresponding to amino acid 442(K) of wild-type fiber was deleted.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 12 - protein, 580 aa, Artificial Sequence
  Codes for a mutated Human Adenovirus type 5 fiber protein. Nucleotides corresponding to amino acid 442(K) of wild-type fiber was deleted.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 13 - DNA, 1746 bp, Artificial Sequence
  Codes for a mutated Human Adenovirus type 5 fiber protein.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgctgcaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 14 - protein, 581 aa, Artificial Sequence
  Codes for a mutated Human Adenovirus type 5 fiber protein.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAAAGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 15 - DNA, 1746 bp, Artificial Sequence
  Codes for a mutated Human Adenovirus type 5 fiber protein.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattatagaattcgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 16 - protein, 581 aa, Artificial Sequence
  Codes for a mutated Human Adenovirus type 5 fiber protein.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIEFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 17 - DNA, 1740 bp, Artificial Sequence
  Codes for a mutated Human Adenovirus type 5 fiber protein. Nucleotides corresponding to amino acids 509(G) & 510(K) of wild-type fiber were deleted.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 18 - protein, 579 aa, Artificial Sequence
  Codes for a mutated Human Adenovirus type 5 fiber protein. Nucleotides corresponding to amino acids 509(G) & 510(K) of wild-type fiber were deleted.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHTAKSNIVSQVYLNGDKTKPVTLTITLNGTQETGDTTPSAYSMSFSWDWSGHNYINEIFATSSYTFSYIAQE

SEQ ID NO: 19 - DNA, 1740 bp, Artificial Sequence
  Codes for a mutated Human Adenovirus type 5 fiber protein. Nucleotides corresponding to amino acids 538(G) & 539(T) of wild-type fiber were deleted.
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaaccaggaaacaggagacacaactccaagtgcatactctatgtcattttcatgggactggtctggccacaactacattaatgaaatatttgccacatcctcttacactttttcatacattgcccaagaataa

SEQ ID NO: 20 - protein, 579 aa, Artificial Sequence
  Codes for a mutated Human Adenovirus type 5 fiber protein. Nucleotides corresponding to amino acids 538(G) & 539(T) of wild-type fiber were deleted.
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNQETGDTTPSAYSMSFSWDWSGHNYINEIFATSSYTFSYIAQE

SEQ ID NO: 21 - DNA, 38 bp, Artificial Sequence
  PCR primer
accacaccagctccagaggctaactgtagactaaatgc

SEQ ID NO: 22 - DNA, 38 bp, Artificial Sequence
  PCR primer
gcatttagtctacagttagcctctggagctggtgtgtt

SEQ ID NO: 23 - DNA, 38 bp, Artificial Sequence
  PCR primer
acagtttcagttttggccggcagtttggctccaatatc

SEQ ID NO: 24 - DNA, 38 bp, Artificial Sequence
  PCR primer
gatattggagccaaactgccggccaaaactgaaactgt

SEQ ID NO: 25 - DNA, 36 bp, Artificial Sequence
  PCR primer
acagtttcagttttggctaaaggcagtttggctcca

SEQ ID NO: 26 - DNA, 36 bp, Artificial Sequence
  PCR primer
tggagccaaactgcctttagccaaaactgaaactgt

SEQ ID NO: 27 - DNA, 36 bp, Artificial Sequence
  PCR primer
gtttcagttttggctgttggcagtttggctccaata

SEQ ID NO: 28 - DNA, 36 bp, Artificial Sequence
  PCR primer
tattggagccaaactgccaacagccaaaactgaaac

SEQ ID NO: 29 - DNA, 36 bp, Artificial Sequence
  PCR primer
gtttcagttttggctgctgcaggcagtttggctcca

SEQ ID NO: 30 - DNA, 36 bp, Artificial Sequence
  PCR primer
tggagccaaactgcctgcagcagccaaaactgaaac

SEQ ID NO: 31 - DNA, 36 bp, Artificial Sequence
  PCR primer
gctcatcttattatagaattcgacgaaaatggagtg

SEQ ID NO: 32 - DNA, 36 bp, Artificial Sequence
  PCR primer
cactccattttcgtcgaattctataataagatgagc

SEQ ID NO: 33 - DNA, 39 bp, Artificial Sequence
  PCR primer
gcttatccaaaatctcacactgccaaaagtaacattgtc

SEQ ID NO: 34 - DNA, 39 bp, Artificial Sequence
  PCR primer
gacaatgttacttttggcagtgtgagattttggataagc

SEQ ID NO: 35 - DNA, 35 bp, Artificial Sequence
  PCR primer
ctaaccattacactaaaccaggaaacaggagacac

SEQ ID NO: 36 - DNA, 35 bp, Artificial Sequence
  PCR primer
gtgtctcctgtttcctggtttagtgtaatggttag

SEQ ID NO: 37 - DNA, 33 bp, Artificial Sequence
  PCR primer
ataagatttgacgaaactggagtgctactaaac

SEQ ID NO: 38 - DNA, 33 bp, Artificial Sequence
  PCR primer
gtttagtagcactccagtttcgtcaaatcttat

SEQ ID NO: 39 - DNA, 33 bp, Artificial Sequence
  PCR primer
tttgacgaaaatggacacctactaaacaattcc

SEQ ID NO: 40 - DNA, 33 bp, Artificial Sequence
  PCR primer
ggaattgtttagtaggtgtccagtttcgtcaaa

SEQ ID NO: 41 - DNA, 33 bp, Artificial Sequence
  PCR primer
aacctatcagcttatgcaaaatctcacggtaaa

SEQ ID NO: 42 - DNA, 32 bp, Artificial Sequence
  PCR primer
tttaccgtgagattttgcataagctgataggt

SEQ ID NO: 43 - protein, 10 aa, Artificial Sequence
  cRGD consensus sequence
HCDCRGDCFC